SEQUENCE LISTING (20 sequences)

SEQ ID NO: 1 - DNA, 20 bp, Homo sapiens
tatgggaaggagtcgttgag

SEQ ID NO: 2 - DNA, 20 bp, Homo sapiens
ctgaatcactccttgccaac

SEQ ID NO: 3 - DNA, 20 bp, Homo sapiens
caaaatgatctgactactcc

SEQ ID NO: 4 - DNA, 20 bp, Homo sapiens
tgaccagtgctgtatgtatc

SEQ ID NO: 5 - DNA, 20 bp, Homo sapiens
tctcacctgataccagattc

SEQ ID NO: 6 - DNA, 20 bp, Homo sapiens
tctctccttaatgcctctat

SEQ ID NO: 7 - DNA, 20 bp, Homo sapiens
actgctgcgggttcctaaag

SEQ ID NO: 8 - DNA, 21 bp, Homo sapiens
ggaagattcgattcaacaagg

SEQ ID NO: 9 - DNA, 19 bp, Homo sapiens
ggtaactatgaatctcatc

SEQ ID NO: 10 - DNA, 20 bp, Homo sapiens
aaaaagcagagaaagcaggg

SEQ ID NO: 11 - DNA, 20 bp, Homo sapiens
agatgatctctgagatgccc

SEQ ID NO: 12 - DNA, 20 bp, Homo sapiens
ccagggtttgtctaccaaag

SEQ ID NO: 13 - DNA, 19 bp, Homo sapiens
aatcacttaaaacaagcag

SEQ ID NO: 14 - DNA, 20 bp, Homo sapiens
cacatacctctacctctagc

SEQ ID NO: 15 - DNA, 19 bp, Homo sapiens
ttccctagtggagcagtgg

SEQ ID NO: 16 - DNA, 20 bp, Homo sapiens
ctttcttcgcaaatgggtgg

SEQ ID NO: 17 - DNA, 20 bp, Homo sapiens
gcactcatcgccacttaatg

SEQ ID NO: 18 - DNA, 20 bp, Homo sapiens
gaacagctaaaccagaacag

SEQ ID NO: 19 - DNA, 20 bp, Homo sapiens
atcactcttatttctccccc

SEQ ID NO: 20 - DNA, 20 bp, Homo sapiens
tgagtcactgtgctaaggag

(19) United States
(12) Patent Application Publication
(10) Pub. No.: US 2020/0354797 A1
(43) Pub. Date: Nov. 12, 2020

(54) METHODS OF ASSESSING RISK OF DEVELOPING BREAST CANCER

(71) Applicants: Genetic Technologies Limited, Fitzroy, Victoria (AU); The University of Melbourne, Parkville, Victoria (AU)

(72) Inventors: Richard ALLMAN, Fitzroy, Victoria (AU); Gillian DITE, The University of Melbourne, Victoria (AU); John HOPPER, The University of Melbourne, Victoria (AU)

(21) Appl. No.: 16/754,662

(22) PCT Filed: Oct. 12, 2018
(86) PCT No.: PCT/AU2018/051113

§ 371 Date: Apr. 8, 2020

(30) Foreign Application Priority Data
Oct. 13, 2017 (AU) 2017904153

(51) Int. Cl. C12Q 1/6886; A61B 10/00; G16H 50/30; G16H 10/60; G16H 70/60

(52) U.S. Cl. CPC C12Q 1/6886; A61B 10/0041; G16H 50/30; A61B 5/4312; G16H 70/60; C12Q 2600/156; C12Q 2600/118; G16H 10/60

(57) ABSTRACT

Methods and systems for assessing the risk of a human female subject for developing breast cancer. In particular, the present disclosure relates to combining a first clinical risk assessment, a second clinical assessment based at least on breast density, and a genetic risk assessment, to obtain an improved risk analysis.

### TECHNICAL FIELD

The present disclosure relates to methods and systems for assessing the risk of a human female subject for developing breast cancer. In particular, the present disclosure relates to combining a first clinical risk assessment, a second clinical assessment based at least on breast density, and a genetic risk assessment, to improve risk analysis.

### BACKGROUND OF THE INVENTION

It is estimated that in the USA approximately one in eight women will develop breast cancer in their lifetime. In 2013 it was predicted that over 230,000 women would be diagnosed with invasive breast cancer and almost 40,000 would die from the disease (ACS Breast Cancer Facts & Figures 2013-14). There is therefore a compelling reason to predict which women will develop disease, and to apply measures to prevent it.

A wide body of research has focused on phenotypic risk factors including age, family history, reproductive history, and benign breast disease. Various combinations of these risk factors have been compiled into the two most commonly used risk prediction algorithms; the Gail Model (appropriate for the general population) (also known as the Breast Cancer Risk Assessment Tool: BCRAT) and the Tyrer-Cuzick Model (appropriate for women with a stronger family history).

These risk prediction algorithms rely largely on self-reported clinical information which is usually obtained by questionnaire. In some instances, relevant clinical information is not provided. This is to be expected, as some questions are reliant on memory from decades' past (first menses), while others require a level of medical sophistication on the part of the patient and/or actual pathology reports (atypical hyperplasia). Furthermore, for those entering an answer rather than ‘unknown’, it brings in to the question the accuracy of data set being entered into the algorithm. For example, whether or not atypical hyperplasia was present is an important factor in breast cancer risk assessment (Relative Risk>4.0).

Recent, commercially available tests for assessing the risk of developing breast cancer discuss predicting breast cancer risk by combining clinical and genetic risk scores. However, the first clinical risk assessment components of these tests are subject to the above referenced limitations of self-reported clinical information. Accordingly, there is the need for improved breast cancer risk assessment tests.

### SUMMARY OF THE INVENTION

The present inventors have found that a breast cancer risk model which combines a first clinical risk assessment, a second clinical risk assessment based at least on breast density, and a genetic risk assessment provides improved risk discrimination for assessing a subject's risk of developing breast cancer.

In an aspect, the present invention provides a method for assessing the risk of a human female subject for developing breast cancer comprising:
performing a first clinical risk assessment of the female subject;
performing a second clinical risk assessment of the female subject, wherein the second clinical assessment is based at least on breast density;
performing a genetic risk assessment of the female subject, wherein the genetic risk assessment involves detecting, in a biological sample derived from the female subject, the presence at least two polymorphisms known to be associated with breast cancer; and
combining the first clinical risk assessment, the second clinical risk assessment, and the genetic risk assessment to obtain the overall risk of a human female subject for developing breast cancer.

In an embodiment, the second clinical risk assessment is based only on breast density.

In an embodiment, performing the first clinical risk assessment uses a model selected from a group consisting of the Gail model, the Claus model, Claus Tables, BOADICEA, the Jonker model, the Claus Extended Formula, the Tyrer-Cuzick model, and the Manchester Scoring System. In some embodiments, the first clinical risk assessment is obtained using the Gail model or BOADICEA or the Tyrer-Cuzick model.

In another embodiment, the first clinical risk assessment includes obtaining information from the female on one or more of the following: medical history of breast cancer, ductal carcinoma or lobular carcinoma, age, age of first menstrual period, age at which she first gave birth, family history of breast cancer, results of previous breast biopsies and race/ethnicity. In an embodiment, the first clinical risk assessment is based only on two or all of the female subject's age, family history of breast cancer and ethnicity. In an embodiment, the first clinical risk assessment is based only on the female subject's age and family history of breast cancer.

In an embodiment, the methods described herein comprise detecting the presence of at least three, four, five, six, seven, eight, nine, ten, 20, 30, 40, 50, 60, 70, 80, 100, 120, 140, 160, 180, or 200 polymorphisms known to be associated with breast cancer.

In an embodiment, the polymorphisms are selected from Table 12 or a polymorphism in linkage disequilibrium with one or more thereof.

In an embodiment, the methods described herein comprise detecting at least 50, 80, 100, 150 of the polymorphisms shown in Table 12, or a polymorphism in linkage disequilibrium with one or more thereof. In some embodiments. the methods described herein comprise detecting all of the 203 polymorphisms shown in Table 12, or a polymorphism in linkage disequilibrium with one or more thereof.

In an embodiment, the polymorphisms are selected from Table 6 or a polymorphism in linkage disequilibrium with one or more thereof.

In another embodiment, the methods described herein comprise detecting at least 72 polymorphisms associated with breast cancer, wherein at least 67 of the polymorphisms are selected from Table 7, or a polymorphism in linkage disequilibrium with one or more thereof, and the remaining polymorphisms are selected from Table 6, or a polymorphism in linkage disequilibrium with one or more thereof.

In an embodiment, when the female subject is Caucasian, the methods described herein comprise detecting at least 72 polymorphisms shown in Table 9, or a polymorphism in linkage disequilibrium with one or more thereof. In an embodiment, when the female subject is Caucasian, the methods described herein comprise detecting all of the 77 polymorphisms shown in Table 9, or a polymorphism in linkage disequilibrium with one or more thereof.

In an embodiment, when the female subject is Negroid or African-American, the methods described herein comprise detecting at least 74 polymorphisms shown in Table 10, or a polymorphism in linkage disequilibrium with one or more thereof. In an embodiment, when the female subject is Negroid or African-American, the methods described herein comprise detecting all of the 74 polymorphisms shown in Table 13, or a polymorphism in linkage disequilibrium with one or more thereof.

In an embodiment, when the female subject is Hispanic, the methods described herein comprise detecting at least 71 polymorphisms shown in Table 11, or a polymorphism in linkage disequilibrium with one or more thereof. In an embodiment, when the female subject is Hispanic, the methods described herein comprise detecting all of the 71 polymorphisms shown in Table 14, or a polymorphism in linkage disequilibrium with one or more thereof.

In some embodiments, combining the first clinical risk assessment, the second clinical risk assessment, and the genetic risk assessment comprises multiplying the risk assessments.

In some embodiments, the female is Caucasian.

In an embodiment, if it is determined the subject has a risk of developing breast cancer, the subject is more likely to be responsive oestrogen inhibition than non-responsive.

In an embodiment, the breast cancer is estrogen receptive positive or estrogen receptor negative.

In an embodiment, the overall risk of the subject for developing breast cancer is an absolute risk. An absolute risk is the risk that pertains to a particular subject rather than a population relative risk. Absolute risk can be described as the numerical probability of a human female subject developing breast cancer within a specified period (e.g. 5, 10, 15, 20 or more years) or in the subject's remaining lifetime.

In another aspect, the present invention provides a method for determining the need for routine diagnostic testing of a human female subject for breast cancer comprising assessing the overall risk of the subject for developing breast cancer using the methods described herein.

In an embodiment, a risk score greater than about 20% lifetime risk indicates that the subject should be enrolled in a screening breast MRIc and mammography program.

In another aspect, the present invention provides a method of screening for breast cancer in a human female subject, the method comprising assessing the overall risk of the subject for developing breast cancer using the methods described herein, and routinely screening for breast cancer in the subject if they are assessed as having a risk for developing breast cancer.

In another aspect, the present invention provides a method for determining the need of a human female subject for prophylactic anti-breast cancer therapy comprising assessing the overall risk of the subject for developing breast cancer using the methods described herein.

In an embodiment, a risk score greater than about 1.66% 5-year risk indicates that estrogen receptor therapy should be offered to the subject.

In another aspect, the present invention provides a method for preventing or reducing the risk of breast cancer in a human female subject, the method comprising assessing the overall risk of the subject for developing breast cancer using the methods described herein, and administering an anti-breast cancer therapy to the subject if they are assessed as having a risk for developing breast cancer.

In an embodiment, the therapy inhibits oestrogen.

In another aspect, the present invention provides an anti-breast cancer therapy for use in preventing breast cancer in a human female subject at risk thereof, wherein the subject is assessed as having a risk for developing breast cancer according to the methods described herein.

In another aspect, the present invention provides a method for stratifying a group of human female subject's for a clinical trial of a candidate therapy, the method comprising assessing the individual overall risk of the subject's for developing breast cancer using the methods as described herein, and using the results of the assessment to select subject's more likely to be responsive to the therapy.

In another aspect, the present invention provides a computer implemented method for assessing the risk of a human female subject for developing breast cancer, the method operable in a computing system comprising a processor and a memory, the method comprising:
receiving first clinical risk data, second clinical risk data, and genetic risk data for the female subject, wherein the first clinical risk data, second clinical risk data and genetic risk data were obtained by a method described herein;
processing the data to combine the clinical risk data with the genetic risk data to obtain the risk of a human female subject for developing breast cancer;
outputting the risk of a human female subject for developing breast cancer.

In another aspect, the present invention provides a system for assessing the risk of a human female subject for developing breast cancer comprising:
system instructions for performing a first clinical risk assessment, a second clinical risk assessment and a genetic risk assessment of the female subject as described herein; and
system instructions for combining the first clinical risk assessment, second clinical risk assessment, and the genetic risk assessment to obtain the risk of a human female subject for developing breast cancer.

Any example herein shall be taken to apply mutatis mutandis to any other example unless specifically stated otherwise.

The present disclosure is not to be limited in scope by the specific examples described herein, which are intended for the purpose of exemplification only. Functionally-equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The disclosure is hereinafter described by way of the following non-limiting

Examples and with reference to the accompanying drawings.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., oncology, breast cancer analysis, molecular genetics, risk assessment and clinical studies).

Unless otherwise indicated, the molecular, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

As used herein, the term “about”, unless stated to the contrary, refers to +/−10%, more preferably +/−5%, more preferably +/−1%, of the designated value.

The methods of the present disclosure can be used to assess risk of a human female subject developing breast cancer. As used herein, the term “breast cancer” encompasses any type of breast cancer that can develop in a female subject. For example, the breast cancer may be characterised as Luminal A (ER+ and/or PR+, HER2−, low Ki67), Luminal B (ER+ and/or PR+, HER2+ (or HER2− with high Ki67), Triple negative/basal-like (ER−, PR−, HER2−) or HER2 type (ER−, PR−, HER2+). In another example, the breast cancer may be resistant to therapy or therapies such as alkylating agents, platinum agents, taxanes, vinca agents, anti-estrogen drugs, aromatase inhibitors, ovarian suppression agents, endocrine/hormonal agents, bisphophonate therapy agents or targeted biological therapy agents. As used herein, “breast cancer” also encompasses a phenotype that displays a predisposition towards developing breast cancer in an individual. A phenotype that displays a predisposition for breast cancer, can, for example, show a higher likelihood that the cancer will develop in an individual with the phenotype than in members of a relevant general population under a given set of environmental conditions (diet, physical activity regime, geographic location, etc.).

As used herein, “biological sample” refers to any sample comprising nucleic acids, especially DNA, from or derived from a human patient, e.g., bodily fluids (blood, saliva, urine etc.), biopsy, tissue, and/or waste from the patient. Thus, tissue biopsies, stool, sputum, saliva, blood, lymph, or the like can easily be screened for polymorphisms, as can essentially any tissue of interest that contains the appropriate nucleic acids. In one embodiment, the biological sample is a cheek cell sample. These samples are typically taken, following informed consent, from a patient by standard medical laboratory methods. The sample may be in a form taken directly from the patient, or may be at least partially processed (purified) to remove at least some non-nucleic acid material.

A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations). Other examples include a deletion or insertion of one or more base pairs at the polymorphism locus.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele “negatively” correlates with a trait when it is linked to it and when presence of the allele is an indicator that a trait or trait form will not occur in an individual comprising the allele.

A marker polymorphism or allele is “correlated” or “associated” with a specified phenotype (breast cancer susceptibility, etc.) when it can be statistically linked (positively or negatively) to the phenotype. Methods for determining whether a polymorphism or allele is statistically linked are known to those in the art. That is, the specified polymorphism occurs more commonly in a case population (e.g., breast cancer patients) than in a control population (e.g., individuals that do not have breast cancer). This correlation is often inferred as being causal in nature, but it need not be, simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies (Slatkin and Excoffier, 1996). LD values according to the present disclosure for neighbouring genotypes/loci are selected above 0.1, preferably, above 0.2, more preferable above 0.5, more preferably, above 0.6, still more preferably, above 0.7, preferably, above 0.8, more preferably above 0.9, ideally about 1.0.

Another way one of skill in the art can readily identify polymorphisms in linkage disequilibrium with the polymorphisms of the present disclosure is determining the LOD score for two loci. LOD stands for “logarithm of the odds”, a statistical estimate of whether two genes, or a gene and a disease gene, are likely to be located near each other on a chromosome and are therefore likely to be inherited. A LOD score of between about 2-3 or higher is generally understood to mean that two genes are located close to each other on the chromosome. Various examples of polymorphisms in linkage disequilibrium with the polymorphisms of the present disclosure are shown in Tables 1 to 4. The present inventors have found that many of the polymorphisms in linkage disequilibrium with the polymorphisms of the present disclosure have a LOD score of between about 2-50. Accordingly, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, polymorphisms in linkage disequilibrium with the polymorphisms of the present disclosure can have a specified genetic recombination distance of less than or equal to about 20 centimorgan (cM) or less. For example, 15 cM or less, 10 cM or less, 9 cM or less, 8 cM or less, 7 cM or less, 6 cM or less, 5 cM or less, 4 cM or less, 3 cM or less, 2 cM or less, 1 cM or less, 0.75 cM or less, 0.5 cM or less, 0.25 cM or less, or 0.1 cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, polymorphisms in linkage disequilibrium with the polymorphisms of the present disclosure are within at least 100 kb (which correlates in humans to about 0.1 cM, depending on local recombination rate), at least 50 kb, at least 20 kb or less of each other.

For example, one approach for the identification of surrogate markers for a particular polymorphism involves a simple strategy that presumes that polymorphisms surrounding the target polymorphism are in linkage disequilibrium and can therefore provide information about disease susceptibility. Thus, as described herein, surrogate markers can therefore be identified from publicly available databases, such as HAPMAP, by searching for polymorphisms fulfilling certain criteria which have been found in the scientific community to be suitable for the selection of surrogate marker candidates (see, for example, the legends of Tables 1 to 4).

“Allele frequency” refers to the frequency (proportion or percentage) at which an allele is present at a locus within an individual, within a line or within a population of lines. For example, for an allele “A,” diploid individuals of genotype “AA,”“Aa,” or “aa” have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the minor allele frequency (MAF). MAF refers to the frequency at which the least common allele occurs in a given population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose, e.g., identifying an individual with a specified genotype (e.g., risk of developing breast cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

As used herein, “risk assessment” refers to a process by which a subject's risk of developing breast cancer can be assessed. A risk assessment will typically involve obtaining information relevant to the subject's risk of developing breast cancer, assessing that information, and quantifying the subject's risk of developing breast cancer, for example, by producing a risk score.

As used herein, “Receiver operating characteristic curves” (ROC) refer to a graphical plot of the sensitivity vs. (1-specificity) for a binary classifier system as its discrimination threshold is varied. The ROC can also be represented equivalently by plotting the fraction of true positives (TPR=true positive rate) vs. the fraction of false positives (FPR=false positive rate). Also known as a Relative Operating Characteristic curve, because it is a comparison of two operating characteristics (TPR & FPR) as the criterion changes. ROC analysis provides tools to select possibly optimal models and to discard suboptimal ones independently from (and prior to specifying) the cost context or the class distribution. Methods of using in the context of the disclosure will be clear to those skilled in the art.

As used herein, the phrase “combining the first clinical risk assessment, the second risk assessment, and the genetic risk assessment” refers to any suitable mathematical analysis relying on the results of the assessments. For example, the results of the first clinical risk assessment, the second clinical risk assessment, and the genetic risk assessment may be added, more preferably multiplied.

As used herein, the terms “routinely screening for breast cancer” and “more frequent screening” are relative terms, and are based on a comparison to the level of screening recommended to a subject who has no identified risk of developing breast cancer.

### Clinical Risk Assessment

In an embodiment, the first and/or second clinical risk assessment procedure includes obtaining clinical information from a female subject. In other embodiments these details have already been determined (such as in the subject's medical records).

In an embodiment, the first clinical risk assessment procedure includes obtaining information from the female on one or more of the following: medical history of breast cancer, ductal carcinoma or lobular carcinoma, age, menstrual history such as age of first menstrual period, age at which she first gave birth, family history of breast cancer or other cancer including the age of the relative at the time of diagnosis, results of previous breast biopsies, use of oral contraceptives, body mass index, alcohol consumption history, smoking history, exercise history, diet and race/ethnicity. Examples of clinical risk assessment procedures include, but are not limited to, the Gail Model (Gail et al., 1989, 1999 and 2007; Costantino et al., 1999; Rockhill et al., 2001), the Claus model (Claus et al., 1994 and 1998), Claus Tables, BOADICEA (Antoniou et al., 2002 and 2004), the Jonker Model (Jonker et al., 2003), the Claus Extended Formula (van Asperen et al., 2004), the Tyrer-Cuzick Model (Tyrer et al., 2004), the Manchester Scoring System (Evans et al., 2004), and the like.

In an embodiment, the first clinical risk assessment is obtained using the Gail Model. Such procedures can be used to estimate the 5-year risk or lifetime risk of a human female subject. The Gail Model is a statistical model which forms the basis of a breast cancer risk assessment tool, named after Dr. Mitchell Gail, Senior Investigator in the Biostatistics Branch of NCI's Division of Cancer Epidemiology and Genetics. The model uses a woman's own personal medical history (number of previous breast biopsies and the presence of atypical hyperplasia in any previous breast biopsy specimen), her own reproductive history (age at the start of menstruation and age at the first live birth of a child), and the history of breast cancer among her first-degree relatives (mother, sisters, daughters) to estimate her risk of developing invasive breast cancer over specific periods of time. Data from the Breast Cancer Detection Demonstration Project (BCDDP), which was a joint NCI and American Cancer Society breast cancer screening study that involved 280,000 women aged 35 to 74 years, and from NCI's Surveillance, Epidemiology, and End Results (SEER) Program were used in developing the model. Estimates for African American women were based on data from the Women's Contraceptive and Reproductive Experiences (CARE) Study and from SEER data. CARE participants included 1,607 women with invasive breast cancer and 1,637 without.

The Gail model has been tested in large populations of white women and has been shown to provide accurate estimates of breast cancer risk. In other words, the model has been “validated” for white women. It has also been tested in data from the Women's Health Initiative for African American women, and the model performs well, but may underestimate risk in African American women with previous biopsies. The model has also been validated for Hispanic women, Asian American women and Native American women.

In another embodiment, the first clinical risk assessment is obtained using the Tyrer-Cuzick model. The Tyrer-Cuzick model incorporates both genetic and non-genetic factors (Tyrer et al., 2004). Nonetheless, the Tyrer-Cuzick model is considered separate from the genetic risk assessment outlined in the present disclosure. The Tyrer-Cuzick uses a three-generation pedigree to estimate the likelihood that an individual carries either a BRCA1/BRCA2 mutation or a hypothetical low-penetrance gene. In addition, the model incorporates personal risk factors, such as parity, body mass index, height, and age at menarche, menopause, HRT use, and first live birth.

In another embodiment, the first clinical risk assessment is obtained using the BOADICEA model. The BOADICEA model was designed with the use of segregation analysis in which susceptibility is explained by mutations in BRCA1 and BRCA2 as well as a polygenic component that reflects the multiplicative effect of multiple genes, which individually have small effects on breast cancer risk (Antoniou et al., 2002 and 2004). This algorithm allows for prediction of BRCA1/BRCA2 mutation probabilities and for cancer risk estimation in individuals with a family history of breast cancer.

In another embodiment, the first clinical risk assessment procedure is obtained using the BRCAPRO model. The BRCAPRO Model is a Bayesian model that incorporates published BRCA1 and BRCA2 mutation frequencies. Cancer penetrance in mutation carriers, cancer status (affected, unaffected, unknown) and age of the patient's first-degree and second degree relatives (Parmigiani et al., 1998). This algorithm allows for prediction of BRCA1/BRCA2 mutation probabilities and for cancer risk estimation in individuals with a family history of breast cancer.

In another embodiment, the first clinical risk assessment is obtained using the Claus model. The Claus Model provides an assessment of hereditary risk of developing breast cancer. The model was developed using data from the Cancer and Steroid Hormone Study. The model originally only included data on family history of breast cancer (Claus et al., 1991), but was later updated to include data on family history of ovarian cancer (Claus et al., 1993). In practice, lifetime risk estimates are usually derived from so-called Claus Tables (Claus et al., 1994). The model was further modified to incorporate information on bilateral disease, ovarian cancer, and three or more affected relatives and termed the “Claus Extended Model” (van Asperen et al., 2004).

In an embodiment, the first clinical risk assessment does not take into consideration breast density.

In an embodiment, the first clinical risk assessment at least takes into consideration the age of the female. In another embodiment, the first clinical risk assessment is based only on the female subject's age and family history of breast cancer. In this embodiment, the first clinical risk assessment can optionally also take ethnicity into consideration. Accordingly, in another embodiment, the first clinical risk assessment is based only on the female subject's family history of breast cancer and ethnicity. In another embodiment, the first clinical risk assessment is based only on the female subject's age and ethnicity. In another embodiment, the first clinical risk assessment is based only on the female subject's age, family history of breast cancer and ethnicity.

In an embodiment, the female subject's family history of breast cancer is based only on the female subject's first degree relatives.

In another embodiment, the female subject's family history of breast cancer is based on the female subject's first degree relatives and second degree relatives.

“Family history of breast cancer” is used in the context of the present disclosure to refer to the history of breast cancer amongst the female subject's first and/or second degree relatives. For example, “family history of breast cancer” can be used to refer to the history of breast cancer amongst only first degree relatives. Put another way, the first clinical risk assessment procedure can take into consideration the female subjects family history of breast cancer amongst first degree relatives. In the context of the present disclosure, a “first degree relative” is a family member who shares about 50 percent of their genes with the female subject. Examples of first degree relatives include parents, offspring, and full-siblings. A “second degree relative” is a family member who shares about 25 percent of their genes with the female subject. Examples of second degree relatives include uncles, aunts, nephews, nieces, grandparents, grandchildren, and half-siblings.

In an embodiment, the first clinical risk assessment at least takes into consideration age, number of previous breast biopsies and known history among first degree relatives. In an embodiment, the first clinical risk assessment at least takes into consideration age, number of previous breast biopsies and known history among first and second degree relatives. In an embodiment, the first clinical risk assessment does not take into consideration third degree or more distant relatives.

In an embodiment, the first clinical risk assessment is based only on the age of the female subject and known history of breast cancer among first degree relatives. In another embodiment, the first clinical risk assessment is based on the age of the female subject, known history of breast cancer among first degree relatives and ethnicity.

As used herein, “based on” means that values are assigned to, for example, the subject's age and family history of breast cancer, but then any suitable calculations are conducted to determine clinical risk.

Clinical information can be self-reported by the female subject. For example, the subject may complete a questionnaire designed to obtain clinical information such as age, history of breast cancer among first degree relatives and ethnicity. In another example, subject to obtaining informed consent from the female subject, clinical information can be obtained from medical records by interrogating a relevant database comprising the clinical information.

In an embodiment, the first clinical risk assessment procedure provides an estimate of the risk of the human female subject developing breast cancer during the next 5-year period (i.e. 5-year risk).

In another embodiment, the first clinical risk assessment procedure provides an estimate of the risk of the human female subject developing breast cancer up to age 90 (i.e. lifetime risk).

In another embodiment, performing the first clinical risk assessment uses a model which calculates the absolute risk of developing breast cancer. For example, the absolute risk of developing breast cancer can be calculated using cancer incidence rates while accounting for the competing risk of dying from other causes apart from breast cancer.

In an embodiment, the first clinical risk assessment provides a 5-year absolute risk of developing breast cancer. In another embodiment, the first clinical risk assessment provides a 10-year absolute risk of developing breast cancer.

The second clinical risk assessment is at least based on breast density. In an embodiment, the second clinical risk assessment is only based on breast density.

Breast density can be measured using any method known in the art. For example, breast density can be estimated based on radiographic appearance of the breast on a mammogram. As will be known to those skilled in the art, dense breast tissue appears light on a mammogram and comprises epithelial and stromal tissue whereas non-dense tissue, comprising fat, appears dark. Thus, in some embodiments, breast density is assessed using a mammogram.

In an embodiment, breast density is assessed using higher pixel brightness thresholds.

In an embodiment, breast density is assessed using percent dense area. Percent dense area is calculated by dividing the area of dense breast tissue by the total breast area identified in a breast image, for example in a mammogram.

In an embodiment, breast density is assessed using Cumulus percent dense area. In another embodiment breast density is assessed using Cumulus percent dense area and non-dense area. “Cumulus” is a software package for semi-automated measurement of dense area from mammograms and is described in (Byng et al., 1994).

In an embodiment, breast density is assessed using a BI-RADS score. “BI-RADS” is an acronym for Breast Imaging-Reporting and Data System, which is a system of standardized numerical codes typically assigned by a radiologist after interpreting a mammogram and is used to communicate a subject's risk of developing breast cancer. A BI-RADS score can also be obtained using automated computerised methods. Typical BI-RADS Assessment Categories (BI-RADS Atlas) are:
0: Incomplete;
1: Negative;
2: Benign;
3: Probably benign;
4: Suspicious;
5: Highly suggestive of malignancy; and
6: Known biopsy—proven malignancy.

### Genetic Risk Assessment

In an embodiment, the genetic risk assessment is performed by analysing the genotype of the subject at 2 or more loci for polymorphisms associated with breast cancer. Various exemplary polymorphisms associated with breast cancer are discussed in the present disclosure. These polymorphisms vary in terms of penetrance and many would be understood by those of skill in the art to be low penetrance polymorphisms.

The term “penetrance” is used in the context of the present disclosure to refer to the frequency at which a particular polymorphism manifests itself within female subjects with breast cancer. “High penetrance” polymorphisms will almost always be apparent in a female subject with breast cancer while “low penetrance” polymorphisms will only sometimes be apparent. In an embodiment polymorphisms assessed as part of a genetic risk assessment according to the present disclosure are low penetrance polymorphisms.

As the skilled addressee will appreciate, each polymorphism which increases the risk of developing breast cancer has an odds ratio of association with breast cancer of greater than 1.0. In an embodiment, the odds ratio is greater than 1.02. Each polymorphism which decreases the risk of developing breast cancer has an odds ratio of association with breast cancer of less than 1.0. In an embodiment, the odds ratio is less than 0.98. Examples of such polymorphisms include, but are not limited to, those provided in Tables 6 to 14, or a polymorphism in linkage disequilibrium with one or more thereof. In an embodiment the genetic risk assessment involves assessing polymorphisms associated with increased risk of developing breast cancer. In another embodiment, the genetic risk assessment involves assessing polymorphisms associated with decreased risk of developing breast cancer. In another embodiment, the genetic risk assessment involves assessing polymorphisms associated with an increased risk of developing breast cancer and polymorphisms associated with a decreased risk of developing breast cancer.

In an embodiment, the genetic risk assessment is performed by analysing the genotype of the subject at two, three, four, five, six, seven, eight, nine, 10 or more loci for polymorphisms associated with breast cancer. Exemplary, polymorphisms relevant for the assessment of breast cancer risk include rs2981582, rs3803662, rs889312, rs13387042, rs13281615, rs4415084, rs3817198, rs4973768, rs6504950 and rs11249433, or a polymorphism in linkage disequilibrium with one or more thereof.

In another embodiment, the genetic risk assessment is performed by analysing the genotype of the subject at 20, 30, 40, 50, 60, 70, 80, 100, 120, 140, 160, 180, 200 or more loci for polymorphisms associated with breast cancer.

In an embodiment, the genetic risk assessment is performed by analysing the genotype of the subject at 72 or more loci for polymorphisms associated with breast cancer. In an embodiment, the genetic risk assessment is performed by analysing the genotype of the subject at 150 or more loci for polymorphisms associated with breast cancer. In an embodiment, the genetic risk assessment is performed by analysing the genotype of the subject at 200 or more loci for polymorphisms associated with breast cancer.

In an embodiment, when performing the methods of the present disclosure to assess risk of breast cancer, at least 67 of the polymorphisms are selected from Table 7 or a polymorphism in linkage disequilibrium with one or more thereof and the remaining polymorphisms are selected from Table 6, or a polymorphism in linkage disequilibrium with one or more thereof. In another embodiment, when performing the methods of the present disclosure at least 68, at least 69, at least 70 of the polymorphisms are selected from Table 7 or a polymorphism in linkage disequilibrium with one or more thereof and the remaining polymorphisms are selected from Table 6, or a polymorphism in linkage disequilibrium with one or more thereof. In one embodiment, at least 72, at least 73, at least 74, at least 75, at least 76, at least 77, at least 78, at least 79, at least 80, at least 81, at least 82, at least 83, at least 84, at least 85, at least 86, at least 87, at least 88 of the polymorphisms shown in Table 6, or a polymorphism in linkage disequilibrium with one or more thereof are assessed. In further embodiments, at least 67, at least 68, at least 69, at least 70 of the polymorphisms shown in Table 7, or a polymorphism in linkage disequilibrium with one or more thereof are assessed. In further embodiments, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88 polymorphisms are assessed, wherein at least 67, at least 68, at least 69, at least 70 polymorphisms shown in Table 7, or a polymorphism in linkage disequilibrium with one or more thereof are assessed, with any remaining polymorphisms being selected from Table 6, or a polymorphism in linkage disequilibrium with one or more thereof.

In some embodiments, when performing the methods of the present disclosure to assess risk of breast cancer, one or more polymorphisms are selected from Table 12 or a polymorphism in linkage disequilibrium with one or more thereof. In an embodiment, at least 50 of the polymorphisms are selected from Table 12, or a polymorphism in linkage disequilibrium with one or more thereof. In an embodiment, at least 60, at least 70, at least 80, at least 90, at least 100, at least 110, at least 120, at least 130, at least 140, at least 150, at least 160, at least 170, at least 180, at least 190, at least 200 of the polymorphisms are selected from Table 12, or a polymorphism in linkage disequilibrium with one or more thereof. In an embodiment, at least 100 of the polymorphisms are selected from Table 12, or a polymorphism in linkage disequilibrium with one or more thereof. In an embodiment, at least 150 of the polymorphisms are selected from Table 12, or a polymorphism in linkage disequilibrium with one or more thereof. In an embodiment, at least 200 of the polymorphisms are selected from Table 12, or a polymorphism in linkage disequilibrium with one or more thereof.

In an embodiment, when determining breast cancer risk, the methods of the present disclosure comprise detecting at least 50, at least 100, or at least 150 polymorphisms shown in Table 12, or a polymorphism in linkage disequilibrium with one or more thereof. In an embodiment, when determining breast cancer risk, the methods of the present disclosure comprise detecting all of the 203 polymorphisms shown in Table 12, or a polymorphism in linkage disequilibrium with one or more thereof.

In an embodiment, when determining breast cancer risk, the methods of the present disclosure comprise detecting at least 50, 80, 100, 150 of the polymorphisms shown in Table 12, or a polymorphism in linkage disequilibrium with one or more thereof.

Polymorphisms in linkage disequilibrium with those specifically mentioned herein are easily identified by those of skill in the art. Examples of such polymorphisms include rs1219648 and rs2420946 which are in strong linkage disequilibrium with rs2981582 (further possible examples provided in Table 1), rs12443621 and rs8051542 which are in strong linkage disequilibrium with polymorphism rs3803662 (further possible examples provided in Table 2), and rs10941679 which is in strong linkage disequilibrium with polymorphism rs4415084 (further possible examples provided in Table 3). In addition, examples of polymorphisms in linkage disequilibrium with rs13387042 are provided in Table 4. Such linked polymorphisms for the other polymorphisms listed in Table 6 or Table 12 can very easily be identified by the skilled person using the HAPMAP database.

TABLE 1
Surrogate markers for polymorphism rs2981582. Markers with a r2 greater than
0.05 to rs2981582 in the HAPMAP dataset (http://hapmap.ncbi.nlm.nih.gov) in
a 1 Mbp interval flanking the marker was selected. Shown is the name of the
correlated polymorphism, values for r2 and D′ to rs2981582 and the corresponding
LOD value, as well as the position of the surrogate marker in NCB Build 36.

| DbSNP | | Correlated | | | | |
|---|---|---|---|---|---|---|
| rsID | Position | SNP | Location | D′ | r² | LOD |
| rs2981582 | 123342307 | rs3135715 | 123344716 | 1.000 | 0.368 | 15.02 |
| rs2981582 | 123342307 | rs7899765 | 123345678 | 1.000 | 0.053 | 2.44 |
| rs2981582 | 123342307 | rs1047111 | 123347551 | 0.938 | 0.226 | 9.11 |
| rs2981582 | 123342307 | rs1219639 | 123348302 | 1.000 | 0.143 | 6.53 |
| rs2981582 | 123342307 | rs10886955 | 123360344 | 0.908 | 0.131 | 5.42 |
| rs2981582 | 123342307 | rs1631281 | 123380775 | 0.906 | 0.124 | 5.33 |
| rs2981582 | 123342307 | rs3104685 | 123381354 | 0.896 | 0.108 | 4.58 |
| rs2981582 | 123342307 | rs1909670 | 123386718 | 1.000 | 0.135 | 6.12 |
| rs2981582 | 123342307 | rs7917459 | 123392364 | 1.000 | 0.135 | 6.42 |
| rs2981582 | 123342307 | rs17102382 | 123393846 | 1.000 | 0.135 | 6.42 |
| rs2981582 | 123342307 | rs10788196 | 123407625 | 1.000 | 0.202 | 9.18 |
| rs2981582 | 123342307 | rs2935717 | 123426236 | 0.926 | 0.165 | 7.30 |
| rs2981582 | 123342307 | rs3104688 | 123426455 | 0.820 | 0.051 | 2.07 |
| rs2981582 | 123342307 | rs4752578 | 123426514 | 1.000 | 0.106 | 5.15 |
| rs2981582 | 123342307 | rs1696803 | 123426940 | 0.926 | 0.168 | 7.33 |
| rs2981582 | 123342307 | rs12262574 | 123428112 | 1.000 | 0.143 | 7.39 |
| rs2981582 | 123342307 | rs4752579 | 123431182 | 1.000 | 0.106 | 5.15 |
| rs2981582 | 123342307 | rs12358208 | 123460953 | 0.761 | 0.077 | 2.46 |
| rs2981582 | 123342307 | rs17102484 | 123462020 | 0.758 | 0.065 | 2.39 |
| rs2981582 | 123342307 | rs2936859 | 123469277 | 0.260 | 0.052 | 1.56 |
| rs2981582 | 123342307 | rs10160140 | 123541979 | 0.590 | 0.016 | 0.40 |

TABLE 2
Surrogate markers for polymorphism rs3803662. Markers with
a r2 greater than 0.05 to rs3803662 in the HAPMAP dataset
(http://hapmap.ncbi.nlm.nih.gov) in a 1 Mbp interval flanking
the marker was selected. Shown is the name of the correlated
polymorphism, values for r2 and D′ to rs3803662 and
the corresponding LOD value, as well as the position of
the surrogate marker in NCB Build 36.

| DbSNP | | Correlated | | | | |
|---|---|---|---|---|---|---|
| rsID | Position | SNP | Location | D′ | r² | LOD |
| rs3803662 | 51143842 | rs4784227 | 51156689 | 0.968 | 0.881 | 31.08 |
| rs3803662 | 51143842 | rs3112572 | 51157948 | 1.000 | 0.055 | 1.64 |
| rs3803662 | 51143842 | rs3104747 | 51159425 | 1.000 | 0.055 | 1.64 |
| rs3803662 | 51143842 | rs3104748 | 51159860 | 1.000 | 0.055 | 1.64 |
| rs3803662 | 51143842 | rs3104750 | 51159990 | 1.000 | 0.055 | 1.64 |
| rs3803662 | 51143842 | rs3104758 | 51166534 | 1.000 | 0.055 | 1.64 |
| rs3803662 | 51143842 | rs3104759 | 51167030 | 1.000 | 0.055 | 1.64 |
| rs3803662 | 51143842 | rs9708611 | 51170166 | 1.000 | 0.169 | 4.56 |
| rs3803662 | 51143842 | rs12935019 | 51170538 | 1.000 | 0.088 | 4.04 |
| rs3803662 | 51143842 | rs4784230 | 51175614 | 1.000 | 0.085 | 4.19 |
| rs3803662 | 51143842 | rs11645620 | 51176454 | 1.000 | 0.085 | 4.19 |
| rs3803662 | 51143842 | rs3112633 | 51178078 | 1.000 | 0.085 | 4.19 |
| rs3803662 | 51143842 | rs3104766 | 51182036 | 0.766 | 0.239 | 7.55 |
| rs3803662 | 51143842 | rs3104767 | 51182239 | 0.626 | 0.167 | 4.88 |
| rs3803662 | 51143842 | rs3112625 | 51183053 | 0.671 | 0.188 | 5.62 |
| rs3803662 | 51143842 | rs12920540 | 51183114 | 0.676 | 0.195 | 5.84 |
| rs3803662 | 51143842 | rs3104774 | 51187203 | 0.671 | 0.188 | 5.62 |
| rs3803662 | 51143842 | rs7203671 | 51187646 | 0.671 | 0.188 | 5.62 |
| rs3803662 | 51143842 | rs3112617 | 51189218 | 0.666 | 0.177 | 5.44 |
| rs3803662 | 51143842 | rs11075551 | 51189465 | 0.666 | 0.177 | 5.44 |
| rs3803662 | 51143842 | rs12929797 | 51190445 | 0.676 | 0.19 | 5.87 |
| rs3803662 | 51143842 | rs3104780 | 51191415 | 0.671 | 0.184 | 5.65 |
| rs3803662 | 51143842 | rs12922061 | 51192501 | 0.832 | 0.631 | 19.14 |
| rs3803662 | 51143842 | rs3112612 | 51192665 | 0.671 | 0.184 | 5.65 |
| rs3803662 | 51143842 | rs3104784 | 51193866 | 0.666 | 0.177 | 5.44 |
| rs3803662 | 51143842 | rs12597685 | 51195281 | 0.671 | 0.184 | 5.65 |
| rs3803662 | 51143842 | rs3104788 | 51196004 | 0.666 | 0.177 | 5.44 |
| rs3803662 | 51143842 | rs3104800 | 51203877 | 0.625 | 0.17 | 4.99 |
| rs3803662 | 51143842 | rs3112609 | 51206232 | 0.599 | 0.163 | 4.86 |
| rs3803662 | 51143842 | rs3112600 | 51214089 | 0.311 | 0.016 | 0.57 |
| rs3803662 | 51143842 | rs3104807 | 51215026 | 0.302 | 0.014 | 0.52 |
| rs3803662 | 51143842 | rs3112594 | 51229030 | 0.522 | 0.065 | 1.56 |
| rs3803662 | 51143842 | rs4288991 | 51230665 | 0.238 | 0.052 | 1.53 |
| rs3803662 | 51143842 | rs3104820 | 51233304 | 0.528 | 0.069 | 1.60 |
| rs3803662 | 51143842 | rs3104824 | 51236594 | 0.362 | 0.067 | 1.93 |
| rs3803662 | 51143842 | rs3104826 | 51237406 | 0.362 | 0.067 | 1.93 |
| rs3803662 | 51143842 | rs3112588 | 51238502 | 0.354 | 0.062 | 1.80 |

TABLE 3
Surrogate markers for polymorphism rs4415084. Markers with
a r2 greater than 0.05 to rs4415084 in the HAPMAP dataset
(http://hapmap.ncbi.nlm.nih.gov) in a 1 Mbp interval flanking
the marker was selected. Shown is the name of the correlated
polymorphism, values for r2 and D′ to rs4415084 and
the corresponding LOD value, as well as the position of
the surrogate marker in NCB Build 36.

| DbSNP | | Correlated | | | | |
|---|---|---|---|---|---|---|
| rsID | Position | SNP | Location | D′ | r² | LOD |
| rs4415084 | 44698272 | rs12522626 | 44721455 | 1.000 | 1.0 | 47.37 |
| rs4415084 | 44698272 | rs4571480 | 44722945 | 1.000 | 0.976 | 40.54 |
| rs4415084 | 44698272 | rs6451770 | 44727152 | 1.000 | 0.978 | 44.88 |
| rs4415084 | 44698272 | rs920328 | 44734808 | 1.000 | 0.893 | 39.00 |
| rs4415084 | 44698272 | rs920329 | 44738264 | 1.000 | 1.0 | 47.37 |
| rs4415084 | 44698272 | rs2218081 | 44740897 | 1.000 | 1.0 | 47.37 |
| rs4415084 | 44698272 | rs16901937 | 44744898 | 1.000 | 0.978 | 45.06 |
| rs4415084 | 44698272 | rs11747159 | 44773467 | 0.948 | 0.747 | 28.79 |
| rs4415084 | 44698272 | rs2330572 | 44776746 | 0.952 | 0.845 | 34.31 |
| rs4415084 | 44698272 | rs994793 | 44779004 | 0.952 | 0.848 | 34.49 |
| rs4415084 | 44698272 | rs1438827 | 44787713 | 0.948 | 0.749 | 29.76 |
| rs4415084 | 44698272 | rs7712949 | 44806102 | 0.948 | 0.746 | 29.19 |
| rs4415084 | 44698272 | rs11746980 | 44813635 | 0.952 | 0.848 | 34.49 |
| rs4415084 | 44698272 | rs16901964 | 44819012 | 0.949 | 0.768 | 30.54 |
| rs4415084 | 44698272 | rs727305 | 44831799 | 0.972 | 0.746 | 27.65 |
| rs4415084 | 44698272 | rs10462081 | 44836422 | 0.948 | 0.749 | 29.76 |
| rs4415084 | 44698272 | rs13183209 | 44839506 | 0.925 | 0.746 | 28.55 |
| rs4415084 | 44698272 | rs13159598 | 44841683 | 0.952 | 0.848 | 34.19 |
| rs4415084 | 44698272 | rs3761650 | 44844113 | 0.947 | 0.744 | 28.68 |
| rs4415084 | 44698272 | rs13174122 | 44846497 | 0.971 | 0.735 | 26.70 |
| rs4415084 | 44698272 | rs11746506 | 44848323 | 0.973 | 0.764 | 29.24 |
| rs4415084 | 44698272 | rs7720787 | 44853066 | 0.952 | 0.845 | 34.31 |
| rs4415084 | 44698272 | rs9637783 | 44855403 | 0.948 | 0.748 | 29.16 |
| rs4415084 | 44698272 | rs4457089 | 44857493 | 0.948 | 0.762 | 29.70 |
| rs4415084 | 44698272 | rs6896350 | 44868328 | 0.948 | 0.764 | 29.46 |
| rs4415084 | 44698272 | rs1371025 | 44869990 | 0.973 | 0.785 | 30.69 |
| rs4415084 | 44698272 | rs4596389 | 44872313 | 0.948 | 0.749 | 29.76 |
| rs4415084 | 44698272 | rs6451775 | 44872545 | 0.948 | 0.746 | 29.19 |
| rs4415084 | 44698272 | rs729599 | 44878017 | 0.948 | 0.748 | 29.16 |
| rs4415084 | 44698272 | rs987394 | 44882135 | 0.948 | 0.749 | 29.76 |
| rs4415084 | 44698272 | rs4440370 | 44889109 | 0.948 | 0.748 | 29.16 |
| rs4415084 | 44698272 | rs7703497 | 44892785 | 0.948 | 0.749 | 29.76 |
| rs4415084 | 44698272 | rs13362132 | 44894017 | 0.952 | 0.827 | 34.09 |
| rs4415084 | 44698272 | rs1438821 | 44894208 | 0.951 | 0.844 | 34.52 |

TABLE 4
Surrogate markers for polymorphism rs13387042. Markers with a r2 greater than
0.05 to rs13387042 in the HAPMAP dataset (http://hapmap.ncbi.nlm.nih.gov) in
a 1 Mbp interval flanking the marker was selected. Shown is the name of the correlated
polymorphism, values for r2 and D′ to rs13387042 and the corresponding LOD
value, as well as the position of the surrogate marker in NCB Build 36.

| DbSNP | | Correlated | | | | |
|---|---|---|---|---|---|---|
| rsID | Position | SNP | Location | D′ | r² | LOD |
| rs13387042 | 217614077 | rs4621152 | 217617230 | 0.865 | 0.364 | 15.30 |
| rs13387042 | 217614077 | rs6721996 | 217617708 | 1.000 | 0.979 | 50.46 |
| rs13387042 | 217614077 | rs12694403 | 217623659 | 0.955 | 0.33 | 14.24 |
| rs13387042 | 217614077 | rs17778427 | 217631258 | 1.000 | 0.351 | 16.12 |
| rs13387042 | 217614077 | rs17835044 | 217631850 | 1.000 | 0.351 | 16.12 |
| rs13387042 | 217614077 | rs7588345 | 217632061 | 1.000 | 0.193 | 8.93 |
| rs13387042 | 217614077 | rs7562029 | 217632506 | 1.000 | 0.413 | 20.33 |
| rs13387042 | 217614077 | rs13000023 | 217632639 | 0.949 | 0.287 | 12.20 |
| rs13387042 | 217614077 | rs13409592 | 217634573 | 0.933 | 0.192 | 7.69 |
| rs13387042 | 217614077 | rs2372957 | 217635302 | 0.855 | 0.168 | 5.97 |
| rs13387042 | 217614077 | rs16856888 | 217638914 | 0.363 | 0.101 | 3.31 |
| rs13387042 | 217614077 | rs16856890 | 217639976 | 0.371 | 0.101 | 3.29 |
| rs13387042 | 217614077 | rs7598926 | 217640464 | 0.382 | 0.109 | 3.60 |
| rs13387042 | 217614077 | rs6734010 | 217643676 | 0.543 | 0.217 | 7.90 |
| rs13387042 | 217614077 | rs13022815 | 217644369 | 0.800 | 0.319 | 12.94 |
| rs13387042 | 217614077 | rs16856893 | 217645298 | 0.739 | 0.109 | 3.45 |
| rs13387042 | 217614077 | rs13011060 | 217646422 | 0.956 | 0.352 | 14.71 |
| rs13387042 | 217614077 | rs4674132 | 217646764 | 0.802 | 0.327 | 13.10 |
| rs13387042 | 217614077 | rs16825211 | 217647249 | 0.912 | 0.326 | 12.95 |
| rs13387042 | 217614077 | rs41521045 | 217647581 | 0.903 | 0.112 | 4.70 |
| rs13387042 | 217614077 | rs2372960 | 217650960 | 0.678 | 0.058 | 2.12 |
| rs13387042 | 217614077 | rs2372967 | 217676158 | 0.326 | 0.052 | 1.97 |
| rs13387042 | 217614077 | rs3843337 | 217677680 | 0.326 | 0.052 | 1.97 |
| rs13387042 | 217614077 | rs2372972 | 217679386 | 0.375 | 0.062 | 2.28 |
| rs13387042 | 217614077 | rs9677455 | 217680497 | 0.375 | 0.062 | 2.28 |
| rs13387042 | 217614077 | rs12464728 | 217686802 | 0.478 | 0.073 | 2.54 |

In another embodiment, when determining breast cancer risk, the methods of the present disclosure encompass assessing all of the polymorphisms shown in Table 6 or Table 12 or a polymorphism in linkage disequilibrium with one or more thereof.

Table 6, Table 7, and Table 12 recite overlapping polymorphisms. It will be appreciated that when selecting polymorphisms for assessment the same polymorphism will not be selected twice. For convenience, the polymorphisms in Table 6 have been separated into Tables 7 and 8. Table 7 lists polymorphisms common across Caucasians, African American and Hispanic populations. Table 8 lists polymorphisms that are not common across Caucasians, African American and Hispanic populations.

In a further embodiment, between 72 and 88, between 73 and 87, between 74 and 86, between 75 and 85, between 76 and 84, between 75 and 83, between 76 and 82, between 77 and 81, between 78 and 80 polymorphisms are assessed, wherein at least 60, at least 61, at least 62, at least 63, at least 64, at least 65, at least 66, at least 67, at least 68, at least 69, at least 70, of the polymorphisms shown in Table 7, or a polymorphism in linkage disequilibrium with one or more thereof are assessed, with any remaining polymorphisms being selected from Table 6, or a polymorphism in linkage disequilibrium with one or more thereof.

In an embodiment, the number of polymorphisms assessed is based on the net reclassification improvement in risk prediction calculated using net reclassification index (NRI) (Pencina et al., 2008).

In an embodiment, the net reclassification improvement of the methods of the present disclosure is greater than 0.01.

In a further embodiment, the net reclassification improvement of the methods of the present disclosure is greater than 0.05.

In yet another embodiment, the net reclassification improvement of the methods of the present disclosure is greater than 0.1.

In another embodiment the genetic risk assessment is performed by analysing the genotype of the subject at 90 or more loci for polymorphisms associated with breast cancer. In another embodiment, the genetic risk assessment is performed by analysing the genotype of the subject at 100, 200, 300, 400, 500, 600, 700, 800, 900, 1,000, 5,000, 10,000, 50,000, 100,000 or more loci for polymorphisms associated with breast cancer. In these embodiments, one or more of the polymorphisms can be selected from Tables 6 to 12.

### Ethnic Genotype Variation

It is known to those of skill in the art that genotypic variation exists between different populations. This phenomenon is referred to as human genetic variation. Human genetic variation is often observed between populations from different ethnic backgrounds. Such variation is rarely consistent and is often directed by various combinations of environmental and lifestyle factors. As a result of genetic variation, it is often difficult to identify a population of genetic markers such as polymorphisms that remain informative across various populations such as populations from different ethnic backgrounds.

A selection of polymorphisms that are common to at least three ethnic backgrounds and remain informative for assessing the risk for developing breast cancer are disclosed herein.

In an embodiment, the methods of the present disclosure can be used for assessing the risk for developing breast cancer in human female subjects from various ethnic backgrounds. For example, the female subject can be classified as Caucasoid, Australoid, Mongoloid and Negroid based on physical anthropology.

In an embodiment, the human female subject can be Caucasian, African American, Hispanic, Asian, Indian, or Latino. In a preferred embodiment, the human female subject is Caucasian, African American or Hispanic. Accordingly, ethnicity can be taken into consideration as part of the clinical and/or genetic risk assessments.

In one embodiment, the human female subject is Caucasian and at least 72, at least 73, at least 74, at least 75, at least 76, at least 77, polymorphisms selected from Table 9, or a polymorphism in linkage disequilibrium with one or more thereof are assessed. Alternatively, all of the 77 polymorphisms selected from Table 9 or a polymorphism in linkage disequilibrium with one or more thereof are assessed.

In another embodiment, the human female subject is Negroid or African American and at least 70, at least 71, at least 72, at least 73, or at least 74 polymorphisms selected from Table 10, or a polymorphism in linkage disequilibrium with one or more thereof are assessed. Alternatively, at least 74 polymorphisms selected from Table 10 or a polymorphism in linkage disequilibrium with one or more thereof are assessed.

In another embodiment, the human female subject is Negroid or African American and at least 70, at least 71, at least 72, at least 73, or at least 74 polymorphisms shown in Table 13, or a polymorphism in linkage disequilibrium with one or more thereof are assessed. In one embodiment, the human female subject is Negroid or African American and the methods described herein comprise detecting all of the 74 polymorphisms shown in Table 13, or a polymorphism in linkage disequilibrium with one or more thereof.

In a further embodiment, the human female subject is Hispanic and at least 67, at least 68, at least 69, at least 70, or at least 71 polymorphisms selected from Table 11, or a polymorphism in linkage disequilibrium with one or more thereof are assessed. Alternatively, at least 71 polymorphisms selected from Table 11 or a polymorphism in linkage disequilibrium with one or more thereof are assessed.

In another embodiment, the human female subject can be Hispanic and at least 67, at least 68, at least 69, at least 70, or at least 71 polymorphisms shown in Table 14, or a polymorphism in linkage disequilibrium with one or more thereof are assessed. In one embodiment, the human female subject can be Hispanic and the methods described herein comprise detecting all of the 71 polymorphisms shown in Table 14, or a polymorphism in linkage disequilibrium with one or more thereof.

It is well known that over time there has been blending of different ethnic origins. However, in practice this does not influence the ability of a skilled person to practice the invention.

A female subject of predominantly European origin, either direct or indirect through ancestry, with white skin is considered Caucasian in the context of the present disclosure. A Caucasian may have, for example, at least 75% Caucasian ancestry (for example, but not limited to, the female subject having at least three Caucasian grandparents).

A female subject of predominantly central or southern African origin, either direct or indirect through ancestry, is considered Negroid in the context of the present disclosure. A Negroid may have, for example, at least 75% Negroid ancestry. An American female subject with predominantly Negroid ancestry and black skin is considered African American in the context of the present disclosure. An African American may have, for example, at least 75% Negroid ancestry. Similar principle applies to, for example, females of Negroid ancestry living in other countries (for example Great Britain, Canada and The Netherlands).

A female subject predominantly originating from Spain or a Spanish-speaking country, such as a country of Central or Southern America, either direct or indirect through ancestry, is considered Hispanic in the context of the present disclosure. An Hispanic may have, for example, at least 75% Hispanic ancestry.

The terms “ethnicity” and “race” can be used interchangeably in the context of the present disclosure. In an embodiment, the genetic risk assessment can readily be practiced based on what ethnicity the subject considers themselves to be. Thus, in an embodiment, the ethnicity of the human female subject is self-reported by the subject. As an example, female subjects can be asked to identify their ethnicity in response to this question: “To what ethnic group do you belong?”. In another example, the ethnicity of the female subject is derived from medical records after obtaining the appropriate consent from the subject or from the opinion or observations of a clinician.

### Calculating Composite Polymorphism Relative Risk “Polymorphism Risk”

An individual's composite polymorphism relative risk score (“Polymorphism risk”) can be defined as the product of genotype relative risk values for each polymorphism assessed. A log-additive risk model can then be used to define three genotypes AA, AB, and BB for a single biallelic polymorphism having relative risk values of 1, OR, and OR², under a rare disease model, where OR is the previously reported disease odds ratio for the high-risk allele, B, vs the low-risk allele, A. If the B allele has frequency (p), then these genotypes have population frequencies of (1−p)², 2p(1−p), and p², assuming Hardy-Weinberg equilibrium. The genotype relative risk values for each polymorphism can then be scaled so that based on these frequencies the average relative risk in the population is 1. Specifically, given the unscaled population average relative risk:

(μ)=(1−p)²+2p(1−p)OR+p²OR²

Adjusted risk values 1/μ, OR/μ, and OR²/μ are used for AA, AB, and BB genotypes. Missing genotypes are assigned a relative risk of 1.

### Combined First Clinical Risk×Second Clinical Risk×Genetic Risk

It is envisaged that the “risk” of a human female subject for developing breast cancer can be provided as a relative risk (or risk ratio) or an absolute risk as required. In an embodiment, the first clinical risk assessment, the second clinical risk assessment, and the genetic risk assessment is combined to obtain the “absolute risk” of a human female subject for developing breast cancer. Absolute risk is the numerical probability of a human female subject developing breast cancer within a specified period (e.g. 5, 10, 15, 20 or more years). It reflects a human female subject's risk of developing breast cancer in so far as it does not consider various risk factors in isolation.

In an embodiment, absolute risk is determined by using any one or more of the following values:
Cumulative incidence of breast cancer from birth to baseline age;
Cumulative incidence of breast cancer from birth to baseline age plus 5 (or 10) years;
Cumulative incidence of breast cancer from birth to age 85 years;
Survival from baseline age to baseline age plus 5 or 10 years; and
Survival from baseline age to age 85 years.

Breast cancer incidence and competing mortality data can be obtained from various sources. For example these data can be obtained from the United States Surveillance, Epidemiology, and End Results Program (SEER) database.

In an embodiment, ethnic-specific breast cancer incidence and competing mortality data are used in the above formula. In an example, ethnic-specific breast cancer incidence and competing mortality data can also be obtained from the SEER database.

Various suitable databases can be used to calculate the relative risk associated with a female subject's family history of breast cancer. One example is provided by the Cancer, Collaborative Group on Hormonal Factors in Breast Cancer (CGoHFiB). In another example, relevant population statistics can be obtained from the Seer database (Siegel et al., 2016).

In another embodiment, the first clinical risk assessment, the second clinical risk assessment, and the genetic risk assessment is combined to obtain the “relative risk” of a human female subject for developing breast cancer. Relative risk (or risk ratio), measured as the incidence of a disease in individuals with a particular characteristic (or exposure) divided by the incidence of the disease in individuals without the characteristic, indicates whether that particular exposure increases or decreases risk. Relative risk is helpful to identify characteristics that are associated with a disease, but by itself is not particularly helpful in guiding screening decisions because the frequency of the risk (incidence) is cancelled out.

### Treatment

After performing the methods of the present disclosure treatment may be prescribed or administered to the subject.

Accordingly, in an embodiment, the methods of the present disclosure relate to an anti-cancer therapy for use in preventing or reducing the risk of breast cancer in a human subject at risk thereof.

One of skill in the art will appreciate that breast cancer is a heterogeneous disease with distinct clinical outcomes (Sorlie et al., 2001). For example, it is discussed in the art that breast cancer may be estrogen receptor positive or estrogen receptor negative. In one embodiment, it is not envisaged that the methods of the present disclosure be limited to assessing the risk of developing a particular type or subtype of breast cancer. For example, it is envisaged that the methods of the present disclosure can be used to assess the risk of developing estrogen receptor positive or estrogen receptor negative breast cancer. In another embodiment, the methods of the present disclosure are used to assess the risk of developing estrogen receptor positive breast cancer. In another embodiment, the methods of the present disclosure are used to assess the risk of developing estrogen receptor negative breast cancer. In another embodiment, the methods of the present disclosure are used to assess the risk of developing metastatic breast cancer. In an example, a therapy that inhibits estrogen is prescribed or administered to the subject.

In another example, a chemopreventative is prescribed or administered to the subject. There are two main classes of drugs currently utilized for breast cancer chemoprevention:
(1) Selective Estrogen Receptor Modulators (SERMs) which block estrogen molecules from binding to their associated cellular receptor. This class of drugs includes for example Tamoxifen and Raloxifene.
(2) Aromatase Inhibitors which inhibit the conversion of androgens into estrogens by the aromatase enzyme Ie reducing the production of estrogens. This class of drugs includes for example Exemestane, Letrozole, Anastrozole, Vorozole, Formestane, Fadrozole.

In an example, a SERM or an aromatase inhibitor is prescribed or administered to the subject.

In an example, Tamoxifen, Raloxifene, Exemestane, Letrozole, Anastrozole, Vorozole, Formestane or Fadrozole is prescribed or administered to a subject.

In an embodiment, the methods of the present disclosure are used to assess the risk of a human female subject for developing breast cancer and administering a treatment appropriate for the risk of developing breast cancer. For example, when performing the methods of the present disclosure indicates a high risk of breast cancer an aggressive chemopreventative treatment regimen can be established. In contrast, when performing the methods of the present disclosure indicates a moderate risk of breast cancer a less aggressive chemopreventative treatment regimen can be established. Alternatively, when performing the methods of the present disclosure indicates a low risk of breast cancer a chemopreventative treatment regimen need not be established. It is envisaged that the methods of the present disclosure can be performed over time so that the treatment regimen can be modified in accordance with the subject's risk of developing breast cancer.

### Marker Detection Strategies

Amplification primers for amplifying markers (e.g., marker loci) and suitable probes to detect such markers or to genotype a sample with respect to multiple marker alleles, can be used in the disclosure. For example, primer selection for long-range PCR is described in U.S. Ser. Nos. 10/042,406 and 10/236,480; for short-range PCR, U.S. Ser. No. 10/341,832 provides guidance with respect to primer selection. Also, there are publicly available programs such as “Oligo” available for primer design. With such available primer selection and design software, the publicly available human genome sequence and the polymorphism locations, one of skill can construct primers to amplify the polymorphisms to practice the disclosure. Further, it will be appreciated that the precise probe to be used for detection of a nucleic acid comprising a polymorphism (e.g., an amplicon comprising the polymorphism) can vary, e.g., any probe that can identify the region of a marker amplicon to be detected can be used in conjunction with the present disclosure. Further, the configuration of the detection probes can, of course, vary. Thus, the disclosure is not limited to the sequences recited herein.

Indeed, it will be appreciated that amplification is not a requirement for marker detection, for example one can directly detect unamplified genomic DNA simply by performing a Southern blot on a sample of genomic DNA.

Typically, molecular markers are detected by any established method available in the art, including, without limitation, allele specific hybridization (ASH), detection of extension, array hybridization (optionally including ASH), or other methods for detecting polymorphisms, amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, and single-strand conformation polymorphisms (SSCP) detection.

Examples of oligonucleotide primers useful for amplifying nucleic acids comprising polymorphisms associated with breast cancer are provided in Table 5. As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

TABLE 5
Examples of oligonucleotide primers useful
for the disclosure.

| Name | Sequence |
|---|---|
| rs889312_for | TATGGGAAGGAGTCGTTGAG |
| | (SEQ ID NO: 1) |
| rs6504950_for | CTGAATCACTCCTTGCCAAC |
| | (SEQ ID NO: 2) |
| rs4973768_for | CAAAATGATCTGACTACTCC |
| | (SEQ ID NO: 3) |
| rs4415084_for | TGACCAGTGCTGTATGTATC |
| | (SEQ ID NO: 4) |
| rs3817198_for | TCTCACCTGATACCAGATTC |
| | (SEQ ID NO: 5) |
| rs3803662_for | TCTCTCCTTAATGCCTCTAT |
| | (SEQ ID NO: 6) |
| rs2981582_for | ACTGCTGCGGGTTCCTAAAG |
| | (SEQ ID NO: 7) |
| rs13387042_for | GGAAGATTCGATTCAACAAGG |
| | (SEQ ID NO: 8) |
| rs13281615_for | GGTAACTATGAATCTCATC |
| | (SEQ ID NO: 9) |
| rs11249433_for | AAAAAGCAGAGAAAGCAGGG |
| | (SEQ ID NO: 10) |
| rs889312_rev | AGATGATCTCTGAGATGCCC |
| | (SEQ ID NO: 11) |
| rs6504950_rev | CCAGGGTTTGTCTACCAAAG |
| | (SEQ ID NO: 12) |
| rs4973768_rev | AATCACTTAAAACAAGCAG |
| | (SEQ ID NO: 13) |
| rs4415084_rev | CACATACCTCTACCTCTAGC |
| | (SEQ ID NO: 14) |
| rs3817198_rev | TTCCCTAGTGGAGCAGTGG |
| | (SEQ ID NO: 15) |
| rs3803662_rev | CTTTCTTCGCAAATGGGTGG |
| | (SEQ ID NO: 16) |
| rs2981582_rev | GCACTCATCGCCACTTAATG |
| | (SEQ ID NO: 17) |
| rs13387042_rev | GAACAGCTAAACCAGAACAG |
| | (SEQ ID NO: 18) |
| rs13281615_rev | ATCACTCTTATTTCTCCCCC |
| | (SEQ ID NO: 19) |
| rs11249433_rev | TGAGTCACTGTGCTAAGGAG |
| | (SEQ ID NO: 20) |

In some embodiments, the primers of the disclosure are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the disclosure be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook et al. (supra).

PCR detection using dual-labelled fluorogenic oligonucleotide probes, commonly referred to as “TaqMan™” probes, can also be performed according to the present disclosure. These probes are composed of short (e.g., 20-25 base) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the “LightCycler®” format described in U.S. Pat. No. 6,174,670.

Array-based detection can be performed using commercially available arrays, e.g., from Affymetrix (Santa Clara, Calif.) or other manufacturers. Reviews regarding the operation of nucleic acid arrays include Sapolsky et al. (1999); Lockhart (1998); Fodor (1997a); Fodor (1997b) and Chee et al. (1996). Array based detection is one preferred method for identification markers of the disclosure in samples, due to the inherently high-throughput nature of array based detection.

The nucleic acid sample to be analysed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

Markers and polymorphisms can also be detected using DNA sequencing. DNA sequencing methods are well known in the art and can be found for example in Ausubel et al, eds., Short Protocols in Molecular Biology, 3rd ed., Wiley, (1995) and Sambrook et al, Molecular Cloning, 2nd ed., Chap. 13, Cold Spring Harbor Laboratory Press, (1989). Sequencing can be carried out by any suitable method, for example, dideoxy sequencing, chemical sequencing, or variations thereof.

Suitable sequencing methods also include Second Generation, Third Generation, or Fourth Generation sequencing technologies, all referred to herein as “next generation sequencing”, including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. A review of some such technologies can be found in (Morozova and Marra, 2008), herein incorporated by reference. Accordingly, in some embodiments, performing a genetic risk assessment as described herein involves detecting the at least two polymorphisms by DNA sequencing. In an embodiment, the at least two polymorphisms are detected by next generation sequencing.

Next generation sequencing (NGS) methods share the common feature of massively parallel, high-throughput strategies, with the goal of lower costs in comparison to older sequencing methods (see, Voelkerding et al., 2009; MacLean et al., 2009).

A number of such DNA sequencing techniques are known in the art, including fluorescence-based sequencing methodologies (Birren et al., 1997). In some embodiments, automated sequencing techniques are used. In some embodiments, parallel sequencing of partitioned amplicons is used (PCT Publication No WO2006084132). In some embodiments, DNA sequencing is achieved by parallel oligonucleotide extension (See, e.g., U.S. Pat. Nos. 5,750,341 and 6,306,597). Additional examples of sequencing techniques include the Church polony technology (Mitra et al., 2003; Shendure et al., 2005; U.S. Pat. Nos. 6,432,36; 6,485,944; 6,511,803), the 454 picotiter pyrosequencing technology (Margulies et al., 2005; U.S. 20050130173), the Solexa single base addition technology (Bennett et al., 2005; U.S. Pat. Nos. 6,787,308; 6,833,246), the Lynx massively parallel signature sequencing technology (Brenner et al., 2000; U.S. Pat. Nos. 5,695,934; 5,714,330), and the Adessi PCR colony technology (Adessi et al., 2000). All documents cited above are incorporated herein by reference.

### Correlating Markers to Phenotypes

These correlations can be performed by any method that can identify a relationship between an allele and a phenotype, or a combination of alleles and a combination of phenotypes. For example, alleles in genes or loci defined herein can be correlated with one or more breast cancer phenotypes. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present disclosure (Hartl et al., 1981). A variety of appropriate statistical models are described in Lynch and Walsh (1998). These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as the use of genetic algorithms, can be used to determine correlations between markers and phenotypes. This is particularly useful when identifying higher order correlations between multiple alleles and multiple phenotypes. To illustrate, neural network approaches can be coupled to genetic algorithm-type programming for heuristic development of a structure-function data space model that determines correlations between genetic information and phenotypic outcomes.

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www.partek.com), e.g., that provide software for pattern recognition (e.g., which provide Partek Pro 2000 Pattern Recognition Software).

Additional details regarding association studies can be found in U.S. Ser. Nos. 10/106,097, 10/042,819, 10/286,417, 10/768,788, 10/447,685, 10/970,761, and U.S. Pat. No. 7,127,355.

Systems for performing the above correlations are also a feature of the disclosure. Typically, the system will include system instructions that correlate the presence or absence of an allele (whether detected directly or, e.g., through expression levels) with a predicted phenotype.

Optionally, the system instructions can also include software that accepts diagnostic information associated with any detected allele information, e.g., a diagnosis that a subject with the relevant allele has a particular phenotype. This software can be heuristic in nature, using such inputted associations to improve the accuracy of the look up tables and/or interpretation of the look up tables by the system. A variety of such approaches, including neural networks, Markov modelling, and other statistical analysis are described above.

### Polymorphic Profiling

The disclosure provides methods of determining the polymorphic profile of an individual at the polymorphisms outlined in the present disclosure (e.g. Table 6 or Table 12) or polymorphisms in linkage disequilibrium with one or more thereof.

The polymorphic profile constitutes the polymorphic forms occupying the various polymorphic sites in an individual. In a diploid genome, two polymorphic forms, the same or different from each other, usually occupy each polymorphic site. Thus, the polymorphic profile at sites X and Y can be represented in the form X (x1, x1), and Y (y1, y2), wherein x1, x1 represents two copies of allele x1 occupying site X and y1, y2 represent heterozygous alleles occupying site Y.

The polymorphic profile of an individual can be scored by comparison with the polymorphic forms associated with resistance or susceptibility to breast cancer occurring at each site. The comparison can be performed on at least, e.g., 1, 2, 5, 10, 25, 50, or all of the polymorphic sites, and optionally, others in linkage disequilibrium with them. The polymorphic sites can be analysed in combination with other polymorphic sites.

Polymorphic profiling is useful, for example, in selecting agents to affect treatment or prophylaxis of breast cancer in a given individual. Individuals having similar polymorphic profiles are likely to respond to agents in a similar way.

Polymorphic profiling is also useful for stratifying individuals in clinical trials of agents being tested for capacity to treat breast cancer or related conditions. Such trials are performed on treated or control populations having similar or identical polymorphic profiles (see EP 99965095.5), for example, a polymorphic profile indicating an individual has an increased risk of developing breast cancer. Use of genetically matched populations eliminates or reduces variation in treatment outcome due to genetic factors, leading to a more accurate assessment of the efficacy of a potential drug.

Polymorphic profiling is also useful for excluding individuals with no predisposition to breast cancer from clinical trials. Including such individuals in the trial increases the size of the population needed to achieve a statistically significant result. Individuals with no predisposition to breast cancer can be identified by determining the numbers of resistances and susceptibility alleles in a polymorphic profile as described above. For example, if a subject is genotyped at ten sites in ten genes of the disclosure associated with breast cancer, twenty alleles are determined in total. If over 50% and alternatively over 60% or 75% percent of these are resistance genes, the individual is unlikely to develop breast cancer and can be excluded from the trial.

In other embodiments, stratifying individuals in clinical trials may be accomplished using polymorphic profiling in combination with other stratification methods, including, but not limited to risk models (e.g., Gail Score, Claus model), clinical phenotypes (e.g., atypical lesions, breast density), and specific candidate biomarkers.

### Computer Implemented Method

It is envisaged that the methods of the present disclosure may be implemented by a system such as a computer implemented method. For example, the system may be a computer system comprising one or a plurality of processors which may operate together (referred to for convenience as “processor”) connected to a memory. The memory may be a non-transitory computer readable medium, such as a hard drive, a solid state disk or CD-ROM. Software, that is executable instructions or program code, such as program code grouped into code modules, may be stored on the memory, and may, when executed by the processor, cause the computer system to perform functions such as determining that a task is to be performed to assist a user to determine the risk of a human female subject for developing breast cancer; receiving data indicating the first clinical risk assessment, the second clinical risk assessment, and the genetic risk assessment of the female subject developing breast cancer, wherein the genetic risk was derived by detecting at least two polymorphisms known to be associated with breast cancer; processing the data to combine the first clinical risk assessment, the second clinical risk assessment, and the genetic risk assessment to obtain the risk of a human female subject for developing breast cancer; outputting the risk of a human female subject for developing breast cancer.

For example, the memory may comprise program code which when executed by the processor causes the system to determine at least two polymorphisms known to be associated with breast cancer; process the data to combine the first clinical risk assessment, the second clinical risk assessment, and the genetic risk assessment to obtain the risk of a human female subject for developing breast cancer; report the risk of a human female subject for developing breast cancer.

In another embodiment, the system may be coupled to a user interface to enable the system to receive information from a user and/or to output or display information. For example, the user interface may comprise a graphical user interface, a voice user interface or a touchscreen.

In an embodiment, the program code may causes the system to determine the “Polymorphism risk”.

In an embodiment, the program code may causes the system to determine Combined First Clinical Risk×Second Clinical Risk×Genetic Risk (for example Polymorphism risk).

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, performing the methods of the present disclosure to assess the risk of a human female subject for developing breast cancer, enables establishment of a diagnostic or prognostic rule based on the the first clinical risk assessment, the second clinical risk assessment and the genetic risk assessment of the female subject developing breast cancer. For example, the diagnostic or prognostic rule can be based on the Combined First Clinical Risk×Second Clinical Risk×Genetic Risk score relative to a control, standard or threshold level of risk. In an embodiment, the threshold level of risk is the level recommended by the

American Cancer Society (ACS) guidelines for screening breast MRIc and mammography. In this example, the threshold level is preferably greater than about (20% lifetime risk).

In another embodiment, the threshold level of risk is the level recommended American Society of Clinical Oncology (ASCO) for offering an estrogen receptor therapy to reduce a subject's risk. In this embodiment, the threshold level of risk is preferably (GAIL index>1.66% for 5-year risk).

In another embodiment, the diagnostic or prognostic rule is based on the application of a statistical and machine learning algorithm. Such an algorithm uses relationships between a population of polymorphisms and disease status observed in training data (with known disease status) to infer relationships which are then used to determine the risk of a human female subject for developing breast cancer in subjects with an unknown risk. An algorithm is employed which provides an risk of a human female subject developing breast cancer. The algorithm performs a multivariate or univariate analysis function.

### Polymorphisms Indicative of Breast Cancer Risk

Examples of polymorphisms indicative of breast cancer risk are shown in Table 6 and Table 12. 77 polymorphisms are informative in Caucasians, 78 polymorphisms are informative in African Americans and 82 are informative in Hispanics. 70 polymorphisms are informative in Caucasians, African Americans and Hispanics (indicated by horizontal stripe pattern; see also Table 7). The remaining 18 polymorphisms (see Table 8) are informative in either Caucasians (indicated by dark trellis pattern; see also Table 9), African Americans (indicated by downward diagonal stripe pattern; see also Table 10) and/or Hispanics (indicated by light grid pattern; see also Table 11). Optimised lists of polymorphisms informative in African Americans and Hispanics are shown in Table 13 and Table 14 respectively.

TABLE 9
Caucasian polymorphisms (n = 77). Alleles represented as major/minor (eg for rs616488
A is the common allele and G less common). OR minor allele numbers below 1 means the minor
allele is not the risk allele, whereas when above 1 the minor allele is the risk allele.

| | | | Minor | OR | | | | | | | |
|---|---|---|---|---|---|---|---|---|---|---|---|
| | | | allele | Minor | | | | | | | |
| polymorphism | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.0661 | 1.05 | AA | 0.95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | CC | 1.07 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rs132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

TABLE 10
African American polymorphisms (n = 78). Alleles represented
as risk/reference (non-risk) (eg for rs616488 A is the risk allele).

| | | | Risk | OR | | | | | | | |
|---|---|---|---|---|---|---|---|---|---|---|---|
| | | | allele | Risk | | | | | | | |
| polymorphism | Chromosome | Alleles | frequency | Allele | μ | Adjusted Risk Score | | | | | |
| rs616488 | 1 | A/G | 0.86 | 1.03 | 1.05 | AA | 0.95 | AG | 0.98 | GG | 1.01 |
| rs11552449 | 1 | C/T | 0.037 | 0.9 | 0.99 | CC | 1.01 | CT | 0.91 | TT | 0.82 |
| rs11249433 | 1 | A/G | 0.13 | 0.99 | 1.00 | AA | 1.00 | AG | 0.99 | GG | 0.98 |
| rs6678914 | 1 | G/A | 0.66 | 1 | 1.00 | GG | 1.00 | GA | 1.00 | AA | 1.00 |
| rs4245739 | 1 | A/C | 0.24 | 0.97 | 0.99 | AA | 1.01 | AC | 0.98 | CC | 0.95 |
| rs12710696 | 2 | G/A | 0.53 | 1.06 | 1.06 | GG | 0.94 | GA | 1.00 | AA | 1.06 |
| rs4849887 | 2 | C/T | 0.7 | 1.16 | 1.24 | CC | 0.81 | CT | 0.94 | TT | 1.09 |
| rs2016394 | 2 | G/A | 0.72 | 1.05 | 1.07 | GG | 0.93 | GA | 0.98 | AA | 1.03 |
| rs1550623 | 2 | A/G | 0.71 | 1.1 | 1.15 | AA | 0.87 | AG | 0.96 | GG | 1.05 |
| rs1045485 | 2 | G/C | 0.93 | 0.99 | 0.98 | GG | 1.02 | GC | 1.01 | CC | 1.00 |
| rs13387042 | 2 | A/G | 0.72 | 1.12 | 1.18 | AA | 0.85 | AG | 0.95 | GG | 1.06 |
| rs16857609 | 2 | C/T | 0.24 | 1.17 | 1.08 | CC | 0.92 | CT | 1.08 | TT | 1.26 |
| rs6762644 | 3 | A/G | 0.46 | 1.05 | 1.05 | AA | 0.96 | AG | 1.00 | GG | 1.05 |
| rs4973768 | 3 | C/T | 0.36 | 1.04 | 1.03 | CC | 0.97 | CT | 1.01 | TT | 1.05 |
| rs12493607 | 3 | G/C | 0.14 | 1.04 | 1.01 | GG | 0.99 | GC | 1.03 | CC | 1.07 |
| rs9790517 | 4 | C/T | 0.084 | 0.88 | 0.98 | CC | 1.02 | CT | 0.90 | TT | 0.79 |
| rs6828523 | 4 | C/A | 0.65 | 1 | 1.00 | CC | 1.00 | CA | 1.00 | AA | 1.00 |
| rs4415084 | 5 | C/T | 0.61 | 1.1 | 1.13 | CC | 0.89 | CT | 0.98 | TT | 1.07 |
| rs10069690 | 5 | C/T | 0.57 | 1.13 | 1.15 | CC | 0.87 | CT | 0.98 | TT | 1.11 |
| rs10941679 | 5 | A/G | 0.21 | 1.04 | 1.02 | AA | 0.98 | AG | 1.02 | GG | 1.06 |
| rs889312 | 5 | A/C | 0.33 | 1.07 | 1.05 | AA | 0.96 | AC | 1.02 | CC | 1.09 |
| rs10472076 | 5 | T/C | 0.28 | 0.95 | 0.97 | TT | 1.03 | TC | 0.98 | CC | 0.93 |
| rs1353747 | 5 | T/G | 0.98 | 1.01 | 1.02 | TT | 0.98 | TG | 0.99 | GG | 1.00 |
| rs1432679 | 5 | A/G | 0.79 | 1.07 | 1.11 | AA | 0.90 | AG | 0.96 | GG | 1.03 |
| rs11242675 | 6 | T/C | 0.51 | 1.06 | 1.06 | TT | 0.94 | TC | 1.00 | CC | 1.06 |
| rs204247 | 6 | A/G | 0.34 | 1.13 | 1.09 | AA | 0.92 | AG | 1.04 | GG | 1.17 |
| rs17529111 | 6 | A/G | 0.075 | 0.99 | 1.00 | AA | 1.00 | AG | 0.99 | GG | 0.98 |
| rs9485370 | 6 | G/T | 0.78 | 1.13 | 1.21 | GG | 0.82 | GT | 0.93 | TT | 1.05 |
| rs3757318 | 6 | G/A | 0.038 | 1.11 | 1.01 | GG | 0.99 | GA | 1.10 | AA | 1.22 |
| rs2046210 | 6 | G/A | 0.6 | 0.99 | 0.99 | GG | 1.01 | GA | 1.00 | AA | 0.99 |
| rs720475 | 7 | G/A | 0.88 | 0.99 | 0.98 | GG | 1.02 | GA | 1.01 | AA | 1.00 |
| rs9693444 | 8 | C/A | 0.37 | 1.06 | 1.04 | CC | 0.96 | CA | 1.01 | AA | 1.08 |
| rs6472903 | 8 | T/G | 0.9 | 1.02 | 1.04 | TT | 0.96 | TG | 0.98 | GG | 1.00 |
| rs2943559 | 8 | A/G | 0.22 | 1.07 | 1.03 | AA | 0.97 | AG | 1.04 | GG | 1.11 |
| rs13281615 | 8 | A/G | 0.43 | 1.06 | 1.05 | AA | 0.95 | AG | 1.01 | GG | 1.07 |
| rs11780156 | 8 | C/T | 0.052 | 0.84 | 0.98 | CC | 1.02 | CT | 0.85 | TT | 0.72 |
| rs1011970 | 9 | G/T | 0.32 | 1.06 | 1.04 | GG | 0.96 | GT | 1.02 | TT | 1.08 |
| rs10759243 | 9 | C/A | 0.59 | 1.02 | 1.02 | CC | 0.98 | CA | 1.00 | AA | 1.02 |
| rs865686 | 9 | T/G | 0.51 | 1.09 | 1.09 | TT | 0.91 | TG | 1.00 | GG | 1.09 |
| rs2380205 | 10 | C/T | 0.42 | 0.98 | 0.98 | CC | 1.02 | CT | 1.00 | TT | 0.98 |
| rs7072776 | 10 | G/A | 0.49 | 1.04 | 1.04 | GG | 0.96 | GA | 1.00 | AA | 1.04 |
| rs11814448 | 10 | A/C | 0.61 | 1.04 | 1.05 | AA | 0.95 | AC | 0.99 | CC | 1.03 |
| rs10822013 | 10 | T/C | 0.23 | 1 | 1.00 | TT | 1.00 | TC | 1.00 | CC | 1.00 |
| rs10995190 | 10 | G/A | 0.83 | 0.98 | 0.97 | GG | 1.03 | GA | 1.01 | AA | 0.99 |
| rs704010 | 10 | C/T | 0.11 | 0.98 | 1.00 | CC | 1.00 | CT | 0.98 | TT | 0.96 |
| rs7904519 | 10 | A/G | 0.78 | 1.13 | 1.21 | AA | 0.82 | AG | 0.93 | GG | 1.05 |
| rs2981579 | 10 | G/A | 0.59 | 1.18 | 1.22 | GG | 0.82 | GA | 0.96 | AA | 1.14 |
| rs2981582 | 10 | G/A | 0.49 | 1.05 | 1.05 | GG | 0.95 | GA | 1.00 | AA | 1.05 |
| rs11199914 | 10 | C/T | 0.48 | 0.97 | 0.97 | CC | 1.03 | CT | 1.00 | TT | 0.97 |
| rs3817198 | 11 | T/C | 0.17 | 0.98 | 0.99 | TT | 1.01 | TC | 0.99 | CC | 0.97 |
| rs3903072 | 11 | G/T | 0.82 | 0.99 | 0.98 | GG | 1.02 | GT | 1.01 | TT | 1.00 |
| rs554219 | 11 | C/G | 0.22 | 1 | 1.00 | CC | 1.00 | CG | 1.00 | GG | 1.00 |
| rs614367 | 11 | G/A | 0.13 | 0.96 | 0.99 | GG | 1.01 | GA | 0.97 | AA | 0.93 |
| rs75915166 | 11 | C/A | 0.015 | 1.44 | 1.01 | CC | 0.99 | CA | 1.42 | AA | 2.05 |
| rs11820646 | 11 | C/T | 0.78 | 0.98 | 0.97 | CC | 1.03 | CT | 1.01 | TT | 0.99 |
| rs12422552 | 12 | G/C | 0.41 | 1.02 | 1.02 | GG | 0.98 | GC | 1.00 | CC | 1.02 |
| rs10771399 | 12 | A/G | 0.96 | 1.19 | 1.40 | AA | 0.72 | AG | 0.85 | GG | 1.01 |
| rs17356907 | 12 | A/G | 0.79 | 1.02 | 1.03 | AA | 0.97 | AG | 0.99 | GG | 1.01 |
| rs1292011 | 12 | A/G | 0.55 | 1.03 | 1.03 | AA | 0.97 | AG | 1.00 | GG | 1.03 |
| rs11571833 | 13 | A/T | 0.003 | 0.95 | 1.00 | AA | 1.00 | AT | 0.95 | TT | 0.90 |
| rs2236007 | 14 | G/A | 0.93 | 0.9 | 0.82 | GG | 1.22 | GA | 1.09 | AA | 0.98 |
| rs999737 | 14 | C/T | 0.95 | 1.03 | 1.06 | CC | 0.95 | CT | 0.97 | TT | 1.00 |
| rs2588809 | 14 | C/T | 0.29 | 1.01 | 1.01 | CC | 0.99 | CT | 1.00 | TT | 1.01 |
| rs941764 | 14 | A/G | 0.7 | 1.1 | 1.14 | AA | 0.87 | AG | 0.96 | GG | 1.06 |
| rs3803662 | 16 | G/A | 0.51 | 0.99 | 0.99 | GG | 1.01 | GA | 1.00 | AA | 0.99 |
| rs17817449 | 16 | T/G | 0.6 | 1.05 | 1.06 | TT | 0.94 | TG | 0.99 | GG | 1.04 |
| rs11075995 | 16 | A/T | 0.18 | 1.07 | 1.03 | AA | 0.98 | AT | 1.04 | TT | 1.12 |
| rs13329835 | 16 | A/G | 0.63 | 1.08 | 1.10 | AA | 0.91 | AG | 0.98 | GG | 1.06 |
| rs6504950 | 17 | G/A | 0.65 | 1.06 | 1.08 | GG | 0.93 | GA | 0.98 | AA | 1.04 |
| rs527616 | 18 | G/C | 0.86 | 0.98 | 0.97 | GG | 1.04 | GC | 1.01 | CC | 0.99 |
| rs1436904 | 18 | T/G | 0.75 | 0.98 | 0.97 | TT | 1.03 | TG | 1.01 | GG | 0.99 |
| rs8170 | 19 | G/A | 0.19 | 1.13 | 1.05 | GG | 0.95 | GA | 1.08 | AA | 1.22 |
| rs4808801 | 19 | A/G | 0.33 | 1.01 | 1.01 | AA | 0.99 | AG | 1.00 | GG | 1.01 |
| rs3760982 | 19 | G/A | 0.47 | 1 | 1.00 | GG | 1.00 | GA | 1.00 | AA | 1.00 |
| rs2284378 | 20 | C/T | 0.16 | 1.06 | 1.02 | CC | 0.98 | CT | 1.04 | TT | 1.10 |
| rs2823093 | 21 | G/A | 0.57 | 1.03 | 1.03 | GG | 0.97 | GA | 1.00 | AA | 1.03 |
| rs132390 | 22 | T/C | 0.052 | 0.88 | 0.99 | TT | 1.01 | TC | 0.89 | CC | 0.78 |
| rs6001930 | 22 | T/C | 0.13 | 1.02 | 1.01 | TT | 0.99 | TC | 1.01 | cc | 1.04 |

TABLE 11
Hispanic polymorphisms (n = 82). Alleles represented as major/minor (eg for rs616488
A is the common allele and G less common). OR minor allele numbers below 1 means the minor
allele is not the risk allele, whereas when above 1 the minor allele is the risk allele.

| | | | Minor | OR | | | | | | | |
|---|---|---|---|---|---|---|---|---|---|---|---|
| | | | allele | Minor | | | | | | | |
| polymorphism | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.0661 | 1.05 | AA | 0.95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | cc | 1.07 |
| rs7696175 | 4 | T/C | 0.38 | 1.14 | 1.11 | TT | 0.90 | CT | 1.03 | cc | 1.17 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs2067980 | 5 | G/A | 0.16 | 1 | 1.00 | GG | 1.00 | AG | 1.00 | AA | 1.00 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs2180341 | 6 | G/A | 0.23 | 0.9600 | 0.98 | GG | 1.02 | AG | 0.98 | AA | 0.94 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs17157903 | 7 | T/C | 0.09 | 0.93 | 0.99 | TT | 1.01 | CT | 0.94 | CC | 0.88 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs2981582 | 10 | T/C | 0.42 | 1.1900 | 1.17 | TT | 0.86 | CT | 1.02 | CC | 1.21 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rs132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

TABLE 12
Expanded list of polymorphisms indicative of
breast cancer risk (n = 203).
“*” indicates a 21 base pair deletion, “**”
indicates a 36 base pair deletion, “***” in-
dicates a 14 base pair insertion, and “****”
indicates a 31 kb deletion. “Odds Ratio minor
allele” values below 1 means that the minor
allele is not the risk allele, whereas when
above 1 the minor allele is the risk allele.

| | | Major | Minor | Odds |
|---|---|---|---|---|
| | | allele/ | allele | ratio |
| | | minor | fre- | minor |
| Polymorphism | Location | allele | quency | allele |
| 12:120832146:C:T | 12q24.31 | C/T | 0.16 | 1.05 |
| 12:85009437:T:C | 12q21.31 | T/C | 0.34 | 0.95 |
| 17:44252468:G:A | 17q21.31 | G/A | 0.19 | 0.95 |
| 4:84370124 | 4q21.23 | TA/TAA | 0.47 | 1.04 |
| chr17:29230520 | 17q11.2 | GGT/G | 0.27 | 0.97 |
| chr22:39359355 | 22q13.1 | I/D**** | 0.10 | 1.10 |
| rs10022462 | 4q22.1 | C/T | 0.44 | 1.04 |
| rs10069690 | 5p15.33 | C/T | 0.26 | 1.06 |
| rs1011970 | 9p21.3 | G/T | 0.16 | 1.07 |
| rs10472076 | 5q11.2 | T/C | 0.38 | 1.03 |
| rs10474352 | 5q14.3 | C/T | 0.16 | 0.94 |
| rs1053338 | 3p14.1 | A/G | 0.14 | 1.05 |
| rs10623258 | 14q32.33 | C/CTT | 0.45 | 1.04 |
| rs10759243 | 9q31.2 | C/A | 0.29 | 1.06 |
| rs10760444 | 9q33.3 | A/G | 0.43 | 1.03 |
| rs10816625 | 9q31.2 | A/G | 0.06 | 1.11 |
| rs10941679 | 5p12 | A/G | 0.25 | 1.15 |
| rs10995201 | 10q21.2 | A/G | 0.16 | 0.90 |
| rs11075995 | 16q12.2 | T/A | 0.24 | 1.03 |
| rs11076805 | 16p13.3 | C/A | 0.26 | 0.97 |
| rs11117758 | 1q41 | G/A | 0.21 | 0.95 |
| rs11199914 | 10q26.12 | C/T | 0.32 | 0.96 |
| rs11242675 | 6p25.3 | T/C | 0.37 | 1.00 |
| rs11249433 | 1p11.2 | A/G | 0.41 | 1.11 |
| rs113577745 | 2p25.1 | C/G | 0.10 | 1.08 |
| rs113701136 | 19q12 | C/T | 0.32 | 1.03 |
| rs11374964 | 11q22.3 | G/GA | 0.42 | 1.00 |
| rs11389348 | 20q12 | G/GT | 0.38 | 0.96 |
| rs11552449 | 1p13.2 | C/T | 0.17 | 1.04 |
| rs11571833 | 13q13.1 | A/T | 0.01 | 1.35 |
| rs116095464 | 5p15.33 | T/C | 0.05 | 1.06 |
| rs11627032 | 14q32.12 | T/C | 0.25 | 0.96 |
| rs117618124 | 18q12.1 | T/C | 0.05 | 0.89 |
| rs11780156 | 8q24.21 | C/T | 0.17 | 1.05 |
| rs11814448 | 10p12.31 | A/C | 0.02 | 1.12 |
| rs11820646 | 11q24.3 | C/T | 0.40 | 0.96 |
| rs11977670 | 7q34 | G/A | 0.43 | 1.06 |
| rs12048493 | 1q21.2 | A/C | 0.38 | 1.04 |
| rs12207986 | 6q14.1 | A/G | 0.47 | 0.97 |
| rs12405132 | 1q21.1 | C/T | 0.37 | 0.97 |
| rs12422552 | 12p13.1 | G/C | 0.26 | 1.06 |
| rs12479355 | 2q36.3 | A/G | 0.21 | 0.96 |
| rs12493607 | 3p24.1 | G/C | 0.34 | 1.05 |
| rs12546444 | 8q23.1 | A/T | 0.10 | 0.93 |
| rs12624860 | 20q13.2 | C/G | 0.24 | 1.04 |
| rs12710696 | 2p24.1 | C/T | 0.37 | 1.03 |
| rs1292011 | 12q24.21 | A/G | 0.42 | 0.92 |
| rs13066793 | 3p12.1 | A/G | 0.09 | 0.94 |
| rs13162653 | 5p15.1 | G/T | 0.45 | 0.99 |
| rs132390 | 22q12.2 | T/C | 0.04 | 1.04 |
| rs13267382 | 8q23.3 | G/A | 0.36 | 1.03 |
| rs13281615 | 8q24.21 | A/G | 0.41 | 1.11 |
| rs13294895 | 9q31.2 | C/T | 0.18 | 1.06 |
| rs13329835 | 16q23.2 | A/G | 0.23 | 1.07 |
| rs13365225 | 8p11.23 | A/G | 0.18 | 0.91 |
| rs1353747 | 5q11.2 | T/G | 0.09 | 0.96 |
| rs140850326 | 1p32.3 | I/D* | 0.49 | 0.97 |
| rs140936696 | 10q23.33 | C/CAA | 0.18 | 1.04 |
| rs1432679 | 5q33.3 | T/C | 0.43 | 1.08 |
| rs1436904 | 18q11.2 | T/G | 0.40 | 0.95 |
| rs151090251 | 15q22.33 | D/I*** | 0.05 | 1.08 |
| rs1550623 | 2q31.1 | A/G | 0.15 | 0.95 |
| rs16857609 | 2q35 | C/T | 0.26 | 1.06 |
| rs16991615 | 20p12.3 | G/A | 0.06 | 1.10 |
| rs1707302 | 1p34.1 | G/A | 0.34 | 0.96 |
| rs17156577 | 7p15.1 | T/C | 0.11 | 1.05 |
| rs17268829 | 7q21.3 | T/C | 0.28 | 1.05 |
| rs17356907 | 12q22 | A/G | 0.30 | 0.91 |
| rs17426269 | 1p22.3 | G/A | 0.15 | 1.05 |
| rs17529111 | 6q14.1 | T/C | 0.22 | 1.02 |
| rs17817449 | 16q12.2 | T/G | 0.41 | 0.95 |
| rs17879961 | 22q12.1 | A/G | 0.005 | 1.26 |
| rs1830298 | 2q33.1 | T/C | 0.28 | 1.06 |
| rs1895062 | 9q33.1 | A/G | 0.41 | 0.94 |
| rs2012709 | 5p13.3 | C/T | 0.48 | 1.02 |
| rs2016394 | 2q31.1 | G/A | 0.47 | 0.95 |
| rs204247 | 6p23 | A/G | 0.44 | 1.04 |
| rs2223621 | 6p22.3 | C/T | 0.38 | 1.04 |
| rs2236007 | 14q13.3 | G/A | 0.21 | 0.93 |
| rs2284378 | 20q11.22 | C/T | 0.32 | 1.00 |
| rs2290203 | 15q26.1 | G/A | 0.21 | 0.94 |
| rs2380205 | 10p15.1 | C/T | 0.44 | 0.98 |
| rs2432539 | 16q13 | G/A | 0.40 | 1.03 |
| rs2588809 | 14q24.1 | C/T | 0.17 | 1.06 |
| rs2594714 | 19p13.12 | G/A | 0.23 | 0.97 |
| rs2747652 | 6q25 | C/T | 0.48 | 0.94 |
| rs2787486 | 17q22 | A/C | 0.30 | 0.93 |
| rs2823093 | 21q21.1 | G/A | 0.27 | 0.94 |
| rs28512361 | 22q13.31 | G/A | 0.11 | 1.05 |
| rs28539243 | 16q12.2 | G/A | 0.49 | 1.05 |
| rs2943559 | 8q21.11 | A/G | 0.08 | 1.10 |
| rs2965183 | 19p13.11 | G/A | 0.35 | 1.04 |
| rs2981578 | 10q26.13 | T/C | 0.47 | 1.23 |
| rs2992756 | 1p36.13 | C/T | 0.49 | 1.06 |
| rs310302 | 8p21.2 | G/A | 0.40 | 1.04 |
| rs3215401 | 5p15.33 | A/AG | 0.31 | 0.93 |
| rs322144 | 19p13.2 | C/G | 0.47 | 0.98 |
| rs34005590 | 2q35 | C/A | 0.05 | 0.82 |
| rs34207738 | 3q23 | CTT/C | 0.41 | 1.06 |
| rs35054928 | 10q26.13 | G/GC | 0.40 | 1.27 |
| rs35383942 | 1q32.1 | C/T | 0.06 | 1.12 |
| rs35951924 | 5q11.1 | A/AT | 0.32 | 0.95 |
| rs36194942 | 18q12.1 | A/AT | 0.30 | 0.98 |
| rs3757322 | 6q25 | T/G | 0.32 | 1.08 |
| rs3760982 | 19q13.31 | G/A | 0.46 | 1.05 |
| rs3817198 | 11p15.5 | T/C | 0.32 | 1.05 |
| rs3819405 | 6p22.3 | C/T | 0.33 | 0.96 |
| rs3903072 | 11q13.1 | G/T | 0.47 | 0.97 |
| rs4233486 | 1p34.2 | T/C | 0.36 | 0.97 |
| rs4245739 | 1q32.1 | A/C | 0.26 | 1.02 |
| rs4442975 | 2q35 | G/T | 0.50 | 0.89 |
| rs4496150 | 16q24.2 | C/A | 0.25 | 0.96 |
| rs4562056 | 5q35.1 | G/T | 0.33 | 1.05 |
| rs45631563 | 10q26.13 | A/T | 0.05 | 0.81 |
| rs4577244 | 2p23.2 | C/T | 0.23 | 1.01 |
| rs4593472 | 7q32.3 | C/T | 0.35 | 0.97 |
| rs4784227 | 16q12.1 | C/T | 0.24 | 1.23 |
| rs4808801 | 19p13.11 | A/G | 0.34 | 0.93 |
| rs4849887 | 2q14.1 | C/T | 0.10 | 0.91 |
| rs4951011 | 1q32.1 | A/G | 0.16 | 1.04 |
| rs4971059 | 1q22 | G/A | 0.35 | 1.05 |
| rs4973768 | 3p24.1 | C/T | 0.50 | 1.11 |
| rs514192 | 8q22.3 | T/A | 0.32 | 1.05 |
| rs527616 | 18q11.2 | G/C | 0.38 | 0.97 |
| rs554219 | 11q13.3 | C/G | 0.13 | 1.21 |
| rs58058861 | 3q26.31 | G/A | 0.21 | 1.06 |
| rs58847541 | 8q24.13 | G/A | 0.15 | 1.08 |
| rs6001930 | 22q13.1 | T/C | 0.10 | 1.12 |
| rs6062356 | 20q13.33 | T/G | 0.15 | 1.06 |
| rs6122906 | 20q13.13 | A/G | 0.18 | 1.05 |
| rs616488 | 1p36.22 | A/G | 0.33 | 0.94 |
| rs62355902 | 5q11.2 | A/T | 0.16 | 1.18 |
| rs6472903 | 8q21.11 | T/G | 0.17 | 0.94 |
| rs6507583 | 18q12.3 | A/G | 0.07 | 0.92 |
| rs6562760 | 13q22.1 | G/A | 0.24 | 0.95 |
| rs6569648 | 6q23.1 | T/C | 0.24 | 0.94 |
| rs6596100 | 5q31.1 | C/T | 0.25 | 0.94 |
| rs6597981 | 11p15 | G/A | 0.48 | 0.96 |
| rs6678914 | 1q32.1 | G/A | 0.41 | 1.00 |
| rs66823261 | 8p23.3 | T/C | 0.22 | 1.03 |
| rs6725517 | 2p23.3 | A/G | 0.41 | 0.96 |
| rs67397200 | 19p13.11 | C/G | 0.30 | 1.03 |
| rs676256 | 9q31.2 | T/C | 0.38 | 0.91 |
| rs6762644 | 3p26.1 | A/G | 0.38 | 1.05 |
| rs67958007 | 10p14 | TG/T | 0.12 | 0.19 |
| rs6796502 | 3p21.31 | G/A | 0.10 | 0.92 |
| rs6805189 | 3p13 | T/C | 0.48 | 0.97 |
| rs6815814 | 4p14 | A/C | 0.26 | 1.06 |
| rs6828523 | 4q34.1 | C/A | 0.12 | 0.91 |
| rs6882649 | 5q22.1 | T/G | 0.34 | 0.97 |
| rs6964587 | 7q21.2 | G/T | 0.39 | 1.03 |
| rs704010 | 10q22.3 | C/T | 0.38 | 1.07 |
| rs7072776 | 10p12.31 | G/A | 0.29 | 1.05 |
| rs71338792 | 19q13.22 | A/AT | 0.23 | 1.05 |
| rs71557345 | 6p22.2 | G/A | 0.07 | 0.92 |
| rs71559437 | 7q22.1 | G/A | 0.12 | 0.93 |
| rs71801447 | 2q13 | CTTATGTT/C | 0.06 | 1.09 |
| rs720475 | 7q35 | G/A | 0.25 | 0.96 |
| rs72749841 | 5q11.1 | T/C | 0.16 | 0.93 |
| rs72755295 | 1q43 | A/G | 0.03 | 1.05 |
| rs72826962 | 17q21.2 | C/T | 0.01 | 1.20 |
| rs7297051 | 12p11.22 | C/T | 0.24 | 0.89 |
| rs73161324 | 22q13.2 | C/T | 0.06 | 1.06 |
| rs738321 | 22q13.1 | C/G | 0.38 | 0.95 |
| rs745570 | 17q25.3 | G/A | 0.50 | 1.03 |
| rs74911261 | 11q22.3 | G/A | 0.03 | 1.01 |
| rs7529522 | 1p12 | T/C | 0.23 | 1.06 |
| rs75915166 | 11q13.3 | C/A | 0.06 | 1.28 |
| rs7707921 | 5q14.2 | A/T | 0.25 | 0.96 |
| rs77528541 | 4q28.1 | G/T | 0.13 | 0.95 |
| rs78269692 | 19p13.13 | T/C | 0.05 | 1.09 |
| rs7904519 | 10q25.2 | A/G | 0.46 | 1.03 |
| rs7971 | 7p15.3 | A/G | 0.35 | 0.96 |
| rs79724016 | 1p34.2 | T/G | 0.03 | 0.93 |
| rs8176636 | 9q34.2 | I/D** | 0.20 | 1.03 |
| rs9257408 | 6p22.1 | G/C | 0.41 | 1.02 |
| rs9348512 | 6p24.3 | C/A | 0.33 | 1.00 |
| rs9358466 | 6p22.3 | T/C | 0.43 | 0.97 |
| rs9397437 | 6q25 | G/A | 0.07 | 1.17 |
| rs941764 | 14q32.11 | A/G | 0.35 | 1.03 |
| rs9485372 | 6q25.1 | G/A | 0.19 | 0.96 |
| rs9693444 | 8p12 | C/A | 0.32 | 1.06 |
| rs9790517 | 4q24 | C/T | 0.23 | 1.04 |
| rs9833888 | 3p12.1 | G/T | 0.22 | 1.06 |
| rs999737 | 14q24.1 | C/T | 0.23 | 0.91 |
| rs2981582 | 10q26 | T/C | | |
| rs3803662 | 16q12.1 | G/A | | |
| rs889312 | 5q11.2 | A/C | | |
| rs13387042 | 2q35 | A/G | | |
| rs4415084 | 5p12 | C/T | | |
| rs1045485 | 2q33.1 | G/C | | |
| rs2736108 | 5p15.33 | C/T | | |
| rs7726159 | 5p15.33 | C/A | | |
| rs12662670 | 6q25.1 | T/G | | |
| rs2046210 | 6q25.1 | G/A | | |
| rs865686 | 9q31.2 | T/G | | |
| rs2981579 | 10q26.13 | G/A | | |
| rs10995190 | 10q21.2 | G/A | | |
| rs78540526 | 11q13.3 | C/T | | |
| rs10771399 | 12p11.22 | A/G | | |
| rs6504950 | 17q22 | G/A | | |
| rs2363956 | 19p13 | G/T | | |
| rs8170 | 19p13.11 | G/A | | |

TABLE 13
Optimised list of African American polymorphisms (n = 74).

| Polymorphism | Chromosome | Major allele/minor allele |
|---|---|---|
| rs11249433 | 1 | G/A |
| rs4245739 | 1 | C/A |
| rs616488 | 1 | A/G |
| rs6678914 | 1 | G/A |
| rs1045485 | 2 | C/G |
| rs12710696 | 2 | A/G |
| rs13387042 | 2 | A/G |
| rs1550623 | 2 | A/G |
| rs16857609 | 2 | A/G |
| rs2016394 | 2 | G/A |
| rs4849887 | 2 | G/A |
| rs12493607 | 3 | G/C |
| rs4973768 | 3 | A/G |
| rs6762644 | 3 | G/A |
| rs6828523 | 4 | C/A |
| rs9790517 | 4 | A/G |
| rs10069690 | 5 | A/G |
| rs10472076 | 5 | G/A |
| rs10941679 | 5 | G/A |
| rs1353747 | 5 | A/C |
| rs1432679 | 5 | G/A |
| rs4415084 | 5 | T/C |
| rs889312 | 5 | C/A |
| rs11242675 | 6 | A/G |
| rs17529111 | 6 | G/A |
| rs204247 | 6 | G/A |
| rs2046210 | 6 | A/G |
| rs3757318 | 6 | A/G |
| rs720475 | 7 | G/A |
| rs11780156 | 8 | A/G |
| rs13281615 | 8 | G/A |
| rs2943559 | 8 | G/A |
| rs6472903 | 8 | A/C |
| rs9693444 | 8 | A/C |
| rs1011970 | 9 | A/C |
| rs10759243 | 9 | A/C |
| rs865686 | 9 | A/C |
| rs10995190 | 10 | G/A |
| rs11199914 | 10 | G/A |
| rs11814448 | 10 | C/A |
| rs2380205 | 10 | G/A |
| rs2981579 | 10 | A/G |
| rs2981582 | 10 | A/G |
| rs704010 | 10 | A/G |
| rs7072776 | 10 | A/G |
| rs7904519 | 10 | G/A |
| rs11820646 | 11 | G/A |
| rs3817198 | 11 | G/A |
| rs3903072 | 11 | C/A |
| rs554219 | 11 | G/C |
| rs614367 | 11 | A/G |
| rs75915166 | 11 | A/C |
| rs10771399 | 12 | A/G |
| rs12422552 | 12 | C/G |
| rs1292011 | 12 | A/G |
| rs17356907 | 12 | A/G |
| rs11571833 | 13 | A/T |
| rs2236007 | 14 | G/A |
| rs2588809 | 14 | A/G |
| rs941764 | 14 | G/A |
| rs999737 | 14 | G/A |
| rs11075995 | 16 | A/T |
| rs13329835 | 16 | G/A |
| rs17817449 | 16 | A/C |
| rs3803662 | 16 | A/G |
| rs6504950 | 17 | G/A |
| rs1436904 | 18 | A/C |
| rs527616 | 18 | C/G |
| rs3760982 | 19 | A/G |
| rs4808801 | 19 | A/G |
| rs8170 | 19 | A/G |
| rs2823093 | 21 | G/A |
| rs132390 | 22 | G/A |
| rs6001930 | 22 | G/A |

TABLE 14
Optimised list of Hispanic polymorphisms (n = 71).

| Polymorphism | Chromosome | Major allele/minor allele |
|---|---|---|
| rs11249433 | 1 | C/T |
| rs11552449 | 1 | T/C |
| rs4245739 | 1 | A/C |
| rs616488 | 1 | G/A |
| rs6678914 | 1 | A/G |
| rs12710696 | 2 | T/C |
| rs13387042 | 2 | A/G |
| rs1550623 | 2 | G/A |
| rs16857609 | 2 | T/C |
| rs2016394 | 2 | A/G |
| rs4849887 | 2 | T/C |
| rs12493607 | 3 | G/G |
| rs4973768 | 3 | T/C |
| rs6762644 | 3 | C/A |
| rs6828523 | 4 | A/C |
| rs7696175 | 4 | T/C |
| rs9790517 | 4 | T/C |
| rs10069690 | 5 | T/C |
| rs10472076 | 5 | C/T |
| rs10941679 | 5 | G/A |
| rs1353747 | 5 | G/T |
| rs1432679 | 5 | C/T |
| rs2067980 | 5 | G/A |
| rs889312 | 5 | C/A |
| rs11242675 | 6 | C/T |
| rs140068132 | 6 | G/A |
| rs204247 | 6 | G/A |
| rs2046210 | 6 | A/G |
| rs2180341 | 6 | G/A |
| rs17157903 | 7 | T/C |
| rs720475 | 7 | A/G |
| rs11780156 | 8 | T/C |
| rs13281615 | 8 | G/A |
| rs2943559 | 8 | G/A |
| rs6472903 | 8 | G/T |
| rs9693444 | 8 | A/C |
| rs1011970 | 9 | T/G |
| rs10759243 | 9 | A/C |
| rs865686 | 9 | T/G |
| rs10995190 | 10 | G/A |
| rs11199914 | 10 | T/C |
| rs11814448 | 10 | C/A |
| rs2380205 | 10 | C/T |
| rs2981579 | 10 | T/C |
| rs2981582 | 10 | T/C |
| rs704010 | 10 | T/C |
| rs7072776 | 10 | A/G |
| rs11820646 | 11 | T/C |
| rs3817198 | 11 | C/T |
| rs3903072 | 11 | T/G |
| rs10771399 | 12 | T/C |
| rs12422552 | 12 | C/G |
| rs1292011 | 12 | A/G |
| rs17356907 | 12 | G/A |
| rs2236007 | 14 | A/G |
| rs2588809 | 14 | T/C |
| rs941764 | 14 | G/A |
| rs999737 | 14 | C/T |
| rs11075995 | 16 | A/T |
| rs13329835 | 16 | G/A |
| rs17817449 | 16 | G/T |
| rs3803662 | 16 | T/C |
| rs6504950 | 17 | G/A |
| rs1436904 | 18 | G/T |
| rs527616 | 18 | C/G |
| rs2363956 | 19 | C/A |
| rs3760982 | 19 | A/G |
| rs4808801 | 19 | G/A |
| rs8170 | 19 | A/G |
| rs2823093 | 21 | G/A |
| rs6001930 | 22 | C/T |

### EXAMPLES

### Example 1

### Combination of a First Clinical Risk Assessment, a Second Clinical Risk Assessment at Least Based on Breast Density, and a Genetic Risk Assessment

The present inventors have found that a breast cancer risk model which combines a first clinical risk assessment, a second clinical risk assessment based at least on breast density, and a genetic risk assessment provides better risk discrimination than any of the currently available individual risk models.

The model has been developed using 800 breast cancer subjects and 2,000 controls and is cross-validated using a second independent cohort comprising 1,259 breast cancer subjects and 1,800 controls.

From a public health perspective, a key issue is how well a risk factor differentiates breast cancer subjects from controls in a given population. This can be determined from the risk gradient, best expressed in terms of the change in odds per adjusted standard deviation (OPERA) of the risk factor in the population about which the inference is being made (Hopper, 2015). OPERA allows risk factors—adjusted for all other factors taken into account by design and analysis, which is the correct way to interpret risk estimates—to be compared for quantitative and binary exposures and thereby puts risk factors into perspective.

The accuracy and clinical validity of the risk scores is determined and validated using approximately 800 breast cancer subjects and 2,000. However, the gold standard in assessing the performance of a new model is a cross-validation in a study population that is independent from that used to build the risk model.

The following specific data fields are included in the model:
A first clinical risk assessment based on age, ethnicity, height, weight, menarche, menopause details, childbirth history, contraceptive usage, hormone replacement therapy usage, family history of breast and ovarian cancer, smoking, alcohol consumption, and mammography history;
A second clinical risk assessment based on breast density measures (Cumulus percent dense area and non-dense area as well as percent density); and
A genetic risk assessment based on genotype data for breast cancer susceptibility loci.

The developed model is “cross-validated” in a second independent cohort of breast cancer subjects and controls. The critical importance of using an independent dataset is to eliminate bias in the estimates of test performance.

### Example 2

### Absolute Risk Estimation

In the case of cancer risk assessment it is often more useful to provide an absolute estimate of cancer risk (ie the risk as it pertains to an individual rather than a population relative risk). The absolute risk is usually described as a remaining lifetime risk or a shorter-term risk such as 5-year risk or 10-year risk (which describe the risk of developing cancer within the next 5 or 10 years respectively).

An absolute risk of developing breast cancer may be derived from the risk model by incorporating the specific incidence of breast cancer in the population under consideration and the competing mortality, which provides an estimate of the risk of dying from causes other than breast cancer.

The following specific data fields can be included in a model to determine the absolute risk of developing breast cancer:
A first clinical risk assessment based on age, ethnicity, height, weight, menarche, menopause details, childbirth history, contraceptive usage, hormone replacement therapy usage, family history of breast and ovarian cancer, smoking, alcohol consumption, and mammography history;
A second clinical risk assessment based on breast density measures (Cumulus percent dense area and non-dense area as well as percent density);
A genetic risk assessment based on genotype data for breast cancer susceptibility loci;
The cumulative incidence of breast cancer from birth to baseline;
The cumulative incidence of breast cancer from birth to baseline plus 5 (or 10) years;
The cumulative incidence of breast cancer from birth to age 85 years;
The survival from baseline age to baseline age plus 5 (or 10) years; and
The survival from baseline age to age 85 years.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU 2017904153 filed 13 Oct. 2017, the entire contents of which are incorporated herein by reference.

### REFERENCES

Adessi et al. (2000) Nucleic Acid Res. 28, E87

Advani and Morena-Aspitia (2014) Breast Cancer: Targets & Therapy; 6: 59-71

Antoniou et al. (2008) Br J Cancer. 98: 1457-1466.

Antoniou et al. (2009) Hum Mol Genet 18: 4442-4456.

American Cancer Society: (2013) Breast Cancer Facts & Figures 2013-1014. Atlanta (Ga.), American Cancer Society Inc, 12.

Bennett et al. (2005) Pharmacogenomics, 6: 373- 382

BI-RADS Atlas (2003) American College of Radiology (ACR) Breast Imaging Reporting and Data System Atlas. Reston, Va: American College of Radiology.

Birren et al. (1997) Genome Analysis: Analyzing DNA, A Laboratory Manual Vol 1.

Brenner et al. (2000) Nat. Biotechnol. 18:630-634

Byng et al. (1994) Phys Med Biol 39:1629-1638.

Cancer, Collaborative Group on Hormonal Factors in Breast Cancer (CGoHFiB) (2001) The Lancet. 358:1389-1399.

Chee et al. (1996) Science 274:610-614.

Chen et al. (2004) Stat Appl Genet Mol Biol. 3: Article 21.

Costantino et al. (1999) J Natl Cancer Inst 91:1541-1548.

De la Cruz (2014) Prim Care Clin Office Pract; 41: 283-306.

Devlin and Risch (1995) Genomics. 29: 311-322.

Biomarkers Prey. pii: cebp.0838.2015.

Fodor (1997a) FASEB Journal 11:A879.

Fodor (1997b) Science 277: 393-395.

Gail et al. (1989) J Natl Cancer Inst 81:1879-1886.

Hartl et al. (1981) A Primer of Population Genetics Washington University, Saint Louis

Hopper (2015) Am J Epidemiol 182:863-7

Shendure et al. (2005) Science 309:1728-1732

Sinauer Associates, Inc. Sunderland, Mass. ISBN: 0-087893-271-2.

Lichtenstein et al. (2000) NEJM 343: 78-85.

Lockhart (1998) Nature Medicine 4:1235-1236.

Lynch and Walsh (1998) Genetics and Analysis of Quantitative Traits, Sinauer Associates, Inc. Sunderland Mass. ISBN 0-87893-481-2.

MacLean et al. Nature Rev. Microbiol, 7:287-296

Mahoney et al. (2008) Cancer J Clin; 58: 347-371.

Margulies et al. (2005) Nature 437: 376-380

Mazzola et al. (2014) Cancer Epidemiol Biomarkers Prey. 23: 1689-1695.

Mealiffe et al. (2010) Natl Cancer Inst. 102: 1618-1627.

Mitra et al. (2003) Analytical Biochemistry 320:55-65

Morozova & Marra (2008) Genomics, 92:255.

Moyer et al. (2013) Ann Intern Med. 159: 698-708.

Parmigiani et al. (1998) Am J Hum Genet. 62: 145-158.

Pencina et al. (2008) Statistics in Medicine 27: 157-172.

Rockhill et al. (2001) J Natl Cancer Inst 93:358-366.

Sapolsky et al. (1999) Genet Anal: Biomolec Engin 14:187-192.

Siegel et al. (2016) Cancer statistics. 66:7-30.

Slatkin and Excoffier (1996) Heredity 76: 377-383.

Sorlie et al. (2001) Proc. Natl. Acad. Sci. 98: 10869-10874.

Tyrer et al. (2004) Stat Med. 23: 1111-1130.

Visvanathan et al. (2009) Journal of Clinical Oncology. 27: 3235-3258.

Voelkerding et al. (2009) Clinical Chem., 55: 641-658

1. A method for assessing the risk of a human female subject for developing breast cancer comprising:
performing a first clinical risk assessment of the female subject;
performing a second clinical risk assessment of the female subject, wherein the second clinical assessment is based at least on breast density;
performing a genetic risk assessment of the female subject, wherein the genetic risk assessment involves detecting, in a biological sample derived from the female subject, the presence at least two polymorphisms known to be associated with breast cancer; and
combining the first clinical risk assessment, the second clinical risk assessment, and the genetic risk assessment to obtain the overall risk of a human female subject for developing breast cancer.

2. The method of claim 1, wherein the second clinical risk assessment is based only on breast density.

3. The method of claim 1 or claim 2, wherein performing the first clinical risk assessment uses a model selected from a group consisting of the Gail model, the Claus model, Claus Tables, BOADICEA, the Jonker model, the Claus Extended Formula, the Tyrer-Cuzick model, and the Manchester Scoring System.

4. The method of claim 3, wherein the first clinical risk assessment is obtained using the Gail model or BOADICEA or the Tyrer-Cuzick model.

5. The method according to any one of claims 1 to 4, wherein performing the first clinical risk assessment includes obtaining information from the female on one or more of the following: medical history of breast cancer, ductal carcinoma or lobular carcinoma, age, age of first menstrual period, age at which she first gave birth, family history of breast cancer, results of previous breast biopsies and race/ethnicity.

6. The method of claim 5, wherein the first clinical risk assessment is based only on two or all of the female subject's age, family history of breast cancer and ethnicity.

7. The method of claim 5 or claim 6, wherein the first clinical risk assessment is based only on the female subject's age and family history of breast cancer.

8. The method according to any one of claims 1 to 7 which comprises detecting the presence of at least three, four, five, six, seven, eight, nine, ten, 20, 30, 40, 50, 60, 70, 80, 100, 120, 140, 160, 180, or 200 polymorphisms known to be associated with breast cancer.

9. The method according to any one of claims 1 to 8, wherein the polymorphisms are selected from Table 12 or a polymorphism in linkage disequilibrium with one or more thereof.

10. The method according to any one of claims 1 to 9, comprising detecting at least 50, 80, 100, 150 of the polymorphisms shown in Table 12, or a polymorphism in linkage disequilibrium with one or more thereof.

11. The method according to any one of claims 1 to 10, comprising detecting all of the 203 polymorphisms shown in Table 12, or a polymorphism in linkage disequilibrium with one or more thereof.

12. The method according to any one of claims 1 to 8, wherein the polymorphisms are selected from Table 6 or a polymorphism in linkage disequilibrium with one or more thereof.

13. The method according to any one of claims 1 to 8, which comprises detecting at least 72 polymorphisms associated with breast cancer, wherein at least 67 of the polymorphisms are selected from Table 7, or a polymorphism in linkage disequilibrium with one or more thereof, and the remaining polymorphisms are selected from Table 6, or a polymorphism in linkage disequilibrium with one or more thereof.

14. The method according to any one of claims 1 to 8, wherein when the female subject is Caucasian, the method comprises detecting at least 72 polymorphisms shown in Table 9, or a polymorphism in linkage disequilibrium with one or more thereof.

15. The method of claim 14, wherein when the female subject is Caucasian, the method comprises detecting all of the 77 polymorphisms shown in Table 9, or a polymorphism in linkage disequilibrium with one or more thereof.

16. The method according to any one of claims 1 to 8, wherein when the female subject is Negroid or African-American, the method comprises detecting at least 74 polymorphisms shown in Table 10, or a polymorphism in linkage disequilibrium with one or more thereof.

17. The method according to any one of claims 1 to 8, wherein when the female subject is Negroid or African-American, the method comprises detecting all of the 74 polymorphisms shown in Table 13, or a polymorphism in linkage disequilibrium with one or more thereof.

18. The method according to any one of claims 1 to 8, wherein when the female subject is Hispanic, the method comprises detecting at least 71 polymorphisms shown in Table 11, or a polymorphism in linkage disequilibrium with one or more thereof.

19. The method according to any one of claims 1 to 8, wherein when the female subject is Hispanic, the method comprises detecting all of the 71 polymorphisms shown in Table 14, or a polymorphism in linkage disequilibrium with one or more thereof.

20. The method according to any one of claims 1 to 19, wherein combining the first clinical risk assessment, the second clinical risk assessment, and the genetic risk assessment comprises multiplying the risk assessments.

21. The method according to any one of claim 1 to 15 or 20, wherein the female is Caucasian.

22. The method according to any one of claims 1 to 21, wherein if it is determined the subject has a risk of developing breast cancer, the subject is more likely to be responsive oestrogen inhibition than non-responsive.

23. The method according to any one of claims 1 to 22, wherein the breast cancer is estrogen receptive positive or estrogen receptor negative.

24. A method for determining the need for routine diagnostic testing of a human female subject for breast cancer comprising assessing the overall risk of the subject for developing breast cancer using the method according to any one of claims 1 to 23.

25. The method of claim 24, wherein a risk score greater than about 20% lifetime risk indicates that the subject should be enrolled in a screening breast MRIc and mammography program.

26. A method of screening for breast cancer in a human female subject, the method comprising assessing the overall risk of the subject for developing breast cancer using the method according to any one of claims 1 to 23, and routinely screening for breast cancer in the subject if they are assessed as having a risk for developing breast cancer.

27. A method for determining the need of a human female subject for prophylactic anti-breast cancer therapy comprising assessing the overall risk of the subject for developing breast cancer using the method according to any one of claims 1 to 23.

28. The method of claim 27, wherein a risk score greater than about 1.66% 5-year risk indicates that estrogen receptor therapy should be offered to the subject.

29. A method for preventing or reducing the risk of breast cancer in a human female subject, the method comprising assessing the overall risk of the subject for developing breast cancer using the method according to any one of claims 1 to 23, and administering an anti-breast cancer therapy to the subject if they are assessed as having a risk for developing breast cancer.

30. The method of claim 29, wherein the therapy inhibits oestrogen.

31. An anti-breast cancer therapy for use in preventing breast cancer in a human female subject at risk thereof, wherein the subject is assessed as having a risk for developing breast cancer according to the method of any one of claims 1 to 23.

32. A method for stratifying a group of human female subject's for a clinical trial of a candidate therapy, the method comprising assessing the individual overall risk of the subject's for developing breast cancer using the method according to any one of claims 1 to 23, and using the results of the assessment to select subject's more likely to be responsive to the therapy.

33. A computer implemented method for assessing the risk of a human female subject for developing breast cancer, the method operable in a computing system comprising a processor and a memory, the method comprising:
receiving first clinical risk data, second clinical risk data, and genetic risk data for the female subject, wherein the first clinical risk data, second clinical risk data and genetic risk data were obtained by a method according to any one of claims 1 to 23;
processing the data to combine the clinical risk data with the genetic risk data to obtain the risk of a human female subject for developing breast cancer;
outputting the risk of a human female subject for developing breast cancer.

34. A system for assessing the risk of a human female subject for developing breast cancer comprising:
system instructions for performing a first clinical risk assessment, a second clinical risk assessment and a genetic risk assessment of the female subject according to any one of claims 1 to 23; and
system instructions for combining the first clinical risk assessment, second clinical risk assessment, and the genetic risk assessment to obtain the risk of a human female subject for developing breast cancer.
